(19) United States
(12) Patent Application Publication
(10) Pub. No.: US 2016/0229835 A1
(43) Pub. Date: Aug. 11, 2016

(54) NOVEL PYRIDYLOXYACETYL TETRAHYDROISOQUINOLINE COMPOUNDS USEFUL AS NAMPT INHIBITORS

(71) Applicant: ELI LILLY AND COMPANY, Indianapolis, IN (US)

(72) Inventors: Timothy Paul Burkholder, Carmel, IN (US); Miriam Filadelfa Del Prado, Madrid (ES); Maria Carmen Fernandez, Madrid (ES); Lawrence Joseph Heinz, II, Pittsboro, IN (US); Lourdes Prieto, Madrid (ES); Genshi Zhao, Carmel, IN (US)

(21) Appl. No.: 15/024,062

(22) PCT Filed: Oct. 3, 2014
(86) PCT No.: PCT/US2014/059054

§ 371 Date: Mar. 23, 2016

(30) Foreign Application Priority Data
Oct. 9, 2013 (EP) 13382399.7

(51) Int. Cl. C07D 401/12; A61K 31/4725; A61K 31/5377; C07D 405/14; A61K 31/496; A61K 31/455; C07D 401/14

(52) U.S. Cl. CPC C07D 401/12; A61K 31/455; A61K 31/4725; C07D 401/14; C07D 405/14; A61K 31/496; A61K 31/5377

(57) ABSTRACT

The present invention provides novel pyridyloxyacetyl tetrahydroisoquinoline compounds that inhibit NAMPT and may be useful in the treatment of cancer.

The present invention relates to novel pyridyloxyacetyl tetrahydroisoquinoline compounds that inhibit activity of nicotinamide phosphoribosyltransferase (NAMPT), pharmaceutical compositions comprising the compounds, and methods of using the compounds to treat physiological disorders, more particularly for the treatment of cancer, during which NAMPT is expressed.

Nicotinamide adenine dinucleotide (NAD⁺) is required for metabolism, energy production, DNA repair, and signaling in many types of cancer cells. In mammals, NAD⁺ can be synthesized from nicotinamide, nicotinic acid or tryptophan. The two-step salvage pathway that converts nicotinamide to NAD⁺ represents the major route to NAD⁺ biosynthesis in mammals.

NAMPT is also essential for the biosynthesis of NAD⁺ in many cancer cells. NAMPT catalyzes the rate-limiting step in the conversion of nictotinamide to nicotinamide mononucleotide (NMN). NAMPT is also found to be upregulated in various cancer cells. Inhibition of NAMPT leads to depletion of NAD⁺. Without sufficient NAD⁺, the synthesis of adenosine-5′-triphosphate (ATP) is inhibited, resulting in eventual attenuation of cancer cell proliferation.

Nicotinic acid phosphoribosyltransferase (NAPRT), an enzyme essential for salvaging NAD⁺ from nicotinic acid is expressed in human tissues and in some tumors. The co-administration of nicotinic acid with certain NAMPT inhibitors has been shown to enhance the therapeutic index since NAD⁺ continues to be synthesized in host tissues from the co-administered nicotinic acid through the NAPRT-mediated nicotinic acid pathway, but as a result, the co-administration of nicotinic acid with these NAMPT inhibitors protects NAPRT-proficient normal cells from the effects of NAMPT inhibitors whereas this co-administration does not appear to affect the antitumor activity of NAMPT inhibitors on NAPRT-deficient tumor cells. This in turn allows an implementation of a rescue strategy in the clinic to enhance the therapeutic index by minimizing potential on-target toxicity of certain NAMPT inhibitors. See also Hasmann, M., et al., Cancer Research 63, 7436-7442, 2003.

NAMPT inhibitors are already known in the art for the treatment of cancer; see for example, FK866/AP0866, disclosed in WO9748696. There are also many other NAMPT inhibitors disclosed in the art, see for example, WO2012038904. There remains a need to provide alternative NAMPT inhibitors, more particularly for the treatment of cancer. Accordingly, the present invention provides NAMPT inhibitors which may be useful for treating cancer.

The present invention provides novel pyridyloxyacetyl tetrahydroisoquinoline compounds that are inhibitors of NAMPT and may have clinical utility as a single agent for treatment of different types of cancers and in particular breast cancer, gastric cancer, colorectal cancer, liver cancer, renal cancer, brain cancer (in particular glioblastoma and neuroblastoma), melanoma, prostate cancer, ovarian cancer, NSCLC, sarcomas (including soft tissues sarcomas), leukemia, lymphoma, endometrial, kidney, adrenal gland, and/or autonomic ganglia cancers.

The present invention provides a compound of the following formula:

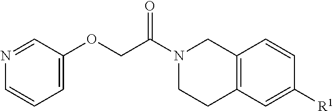

Wherein:

R¹ is —NHSO₂R², —NHC(O)CH₂R³, —CH₂-piperazinyl-C(O)R⁴, or —CH(CH₃)-piperazinyl-C(O)R⁴;

R² is N-methylpiperidin-4-yl, N-oxetan-3-yl-piperidin-4-yl, tetrahydropyran-4-yl, tetrahydropyran-4-yl-N-carbonyl-piperidin-4-yl, 2-hydroxy-2-methyl-prop-1-yl, methoxyethyl, 2-isopropoxyethyl, 2-trifluoromethylethyl, cyclopropylmethyl, or pyrid-2-yl;

R³ is tetrahydropyran-2-yl, t-butyl, —C(CH₃)(CH₃)(OH)—C(OH)(CH₃)(CH₂CH₃), or —C(OH)(CH₃)(CF₃);

R⁴ is tetrahydropyran-4-yl, tetrahydropyran-4-yl-methyl, morpholin-4-yl-methyl, or 2-hydroxy-2-methyl-propyl;

or a pharmaceutically acceptable salt thereof.

Preferably, R¹ is —NHSO₂R².

The present invention provides a compound which is 2-hydroxy-2-methyl-N-[2-[2-(3-pyridyloxy)acetyl]-3,4-dihydro-1H-isoquinolin-6-yl]propane-1-sulfonamide, or a pharmaceutically acceptable salt thereof.

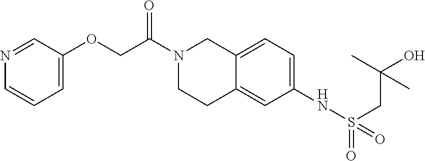 [SMILES: CC(C)(O)CS(=O)(=O)Nc1ccc2c(c1)CCN(C(=O)COc1cccnc1)C2]

The present invention also provides a compound which is 2-methoxy-N-[2-[2-(3-pyridyloxy)acetyl]-3,4-dihydro-1H-isoquinolin-6-yl]ethanesulfonamide, or a pharmaceutically acceptable salt thereof.

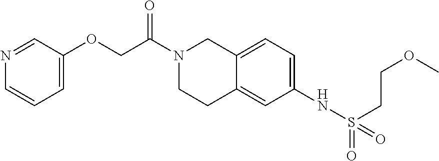 [SMILES: COCCS(=O)(=O)Nc1ccc2c(c1)CCN(C(=O)COc1cccnc1)C2]

The present invention provides a method of treating breast cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating gastric cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating colorectal cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating liver cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating renal cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating brain cancer in a mammal comprising administering to a mammal in need of such treatment an effective amount of a compound or salt of the present invention. In particular, the brain cancer is glioblastoma and neuroblastoma. Optionally, this method further comprises the simultaneous, separate, or sequential administration of nicotinic acid.

The present invention provides a method of treating melanoma in a mammal comprising administering to a mammal in need of such treatment an effective amount of a compound or salt of the present invention. Optionally, this method further comprises the simultaneous, separate, or sequential administration of nicotinic acid.

The present invention provides a method of treating prostate cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating ovarian cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating NSCLC in a mammal comprising administering to a mammal in need of such treatment an effective amount of a compound or salt of the present invention. Optionally, this method further comprises the simultaneous, separate, or sequential administration of nicotinic acid.

The present invention provides a method of treating sarcomas, in particular soft tissue sarcomas, in a mammal comprising administering to a mammal in need of such treatment an effective amount of a compound or salt of the present invention. Optionally, this method further comprises the simultaneous, separate, or sequential administration of nicotinic acid.

The present invention provides a method of treating leukemia in a mammal comprising administering to a mammal in need of such treatment an effective amount of a compound or salt of the present invention. Optionally, this method further comprises the simultaneous, separate, or sequential administration of nicotinic acid.

The present invention provides a method of treating lymphoma in a mammal comprising administering to a mammal in need of such treatment an effective amount of a compound or salt of the present invention. Optionally, this method further comprises the simultaneous, separate, or sequential administration of nicotinic acid.

The present invention provides a method of treating endometrial cancer in a mammal comprising administering to a mammal in need of such treatment an effective amount of a compound or salt of the present invention. Optionally, this method further comprises the simultaneous, separate, or sequential administration of nicotinic acid.

The present invention provides a method of treating kidney cancer in a mammal comprising administering to a mammal in need of such treatment an effective amount of a compound or salt of the present invention. Optionally, this method further comprises the simultaneous, separate, or sequential administration of nicotinic acid.

The present invention provides 2-hydroxy-2-methyl-N-[2-[2-(3-pyridyloxy)acetyl]-3,4-dihydro-1H-isoquinolin-6-yl]propane-1-sulfonamide in crystalline form. The present invention also provides 2-hydroxy-2-methyl-N-[2-[2-(3-pyridyloxy)acetyl]-3,4-dihydro-1H-isoquinolin-6-yl]propane-1-sulfonamide in crystalline anhydrous free base form characterized by a X-ray powder diffraction pattern having characteristic peaks, in 2θ±0.2, occurring at 17.97 and one or more of 21.59, 18.53, and 14.96.

The present invention provides 2-methoxy-N-[2-[2-(3-pyridyloxy)acetyl]-3,4-dihydro-1H-isoquinolin-6-yl]ethanesulfonamide in crystalline form. The present invention also provides 2-methoxy-N-[2-[2-(3-pyridyloxy)acetyl]-3,4-dihydro-1H-isoquinolin-6-yl]ethanesulfonamide in crystalline anhydrous free base form characterized by a X-ray powder diffraction pattern having characteristic peaks, in 2θ±0.2, occurring at 24.21 and one or more of 15.73, 18.95, and 18.28.

The invention also provides pharmaceutical compositions comprising a compound or salt of the present invention and one or more pharmaceutically acceptable carriers, diluents, or excipients. Optionally, the composition further comprises nicotinic acid.

This invention also provides a compound or salt of the present invention for use in therapy. The invention also provides a compound or salt of the present invention for use in the treatment of cancer. Additionally, this invention provides use of a compound or salt of the present invention in the manufacture of a medicament for treating cancer. Additionally, this invention provides a compound or salt of the present invention for use in the treatment of cancer. In particular, this cancer is breast cancer. Additionally, this cancer is gastric cancer. Additionally, this cancer is colorectal cancer. Additionally, this cancer is liver cancer. Additionally, this cancer is renal cancer. Additionally, this cancer is brain cancer, more particularly glioblastoma and neuroblastoma. Additionally, this cancer is melanoma. Additionally, this cancer is prostate cancer. Additionally, this cancer is ovarian cancer. Additionally, this cancer is NSCLC. Additionally, this cancer is sarcoma, more particularly soft tissue sarcoma. Additionally this cancer is leukemia. Additionally, this cancer is lymphoma. Additionally, this cancer is endometrial cancer. Additionally, this cancer is kidney cancer. Additionally, the compound or salt is optionally administered in simultaneous, separate, or sequential combination with nicotinic acid.

It will be understood by the skilled artisan that compounds of the present invention are capable of forming salts. The compounds of the present invention contain basic heterocycles, and accordingly react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008); S. M. Berge, et al., “Pharmaceutical Salts”, Journal of Pharmaceutical Sciences, Vol 66, No. 1, January 1977.

The skilled artisan will appreciate that certain compounds of the present invention contain at least one chiral center. The present invention contemplates all individual enantiomers or diastereomers, as well as mixtures of the enantiomers and diastereomers of said compounds including racemates. It is preferred that compounds of the present invention containing at least one chiral center exist as single enantiomers or diastereomers. The single enantiomers or diastereomers may be prepared beginning with chiral reagents or by stereoselective or stereospecific synthetic techniques. Alternatively, the single enantiomers or diastereomers may be isolated from mixtures by standard chiral chromatographic or crystallization techniques.

The compounds of the present invention can be prepared according to synthetic methods well known and appreciated in the art. Suitable reaction conditions for the steps of these reactions are well known in the art and appropriate substitutions of solvents and co-reagents are within the skill of the art. Likewise, it will be appreciated by those skilled in the art that synthetic intermediates may be isolated and/or purified by various well known techniques as needed or desired, and that frequently, it will be possible to use various intermediates directly in subsequent synthetic steps with little or no purification. Furthermore, the skilled artisan will appreciate that in some circumstances, the order in which moieties are introduced is not critical. The particular order of steps required to produce the compounds of the present invention is dependent upon the particular compound being synthesized, the starting compound, and the relative liability of the substituted moieties, as is well appreciated by the skilled chemist. All substituents, unless otherwise indicated, are as previously defined, and all reagents are well known and appreciated in the art.

Unless noted to the contrary, the compounds illustrated herein are named and numbered using either ACDLABS or Accelrys Draw 4.0.

As used herein, the following terms have the meanings indicated: “ACN” refers to acetonitrile; “ATP” refers to adenosine-5′-triphosphate; “BID” refers to twice a day; “BOC” refers to di-tert-butyl-dicarbonate; “DMSO” refers to dimethylsulfoxide; “DTT” refers to dithiothreitol; “FBS” refers to fetal bovine serum; “HATU” refers to O-(7-azabenzotriazol-1-yl)-N,N,N′,N′-tetramethyluronium hexafluorophosphate; “HEPES” refers to 4-(2-hydroxyethyl)-1-piperazineethanesulfonic acid; “HPLC” refers to high-pressure liquid chromatography; “Isomer 2” refers to the isomer that elutes off of the column second; “IVTI” refers to in vivo target inhibition; “LC” refers to liquid chromatography; “MS” refers to mass spectroscopy; “NAD⁺” refers to nicotinamide adenine dinucleotide; “NADH” refers to reduce nicotinamide adenine dinucleotide; “NAMPT” refers to nicotinamide phosphoribosyltransferase; “NAPRT” refers to nicotinic acid phosphoribosyltransferase; “NMN” refers to nicotinamide mononucleotide; “NMR” refers to nuclear magnetic resonance; “NSCLC” refers to non-small cell lung cancer; “SD” refers to standard deviation; “SE” refers for standard error.

Compounds of the present invention may be synthesized as illustrated in the following schemes, where R¹ to R⁴ are as previously defined.

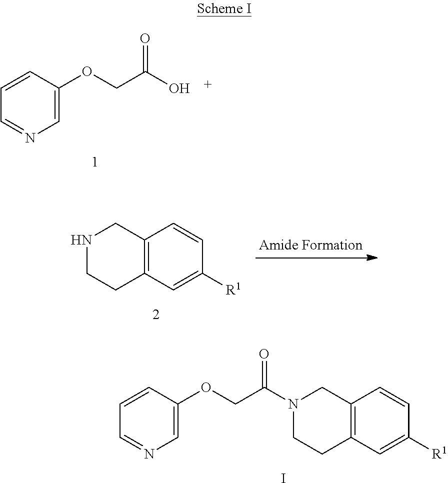

Amide formation to make the compound of Formula I

The compounds of the present invention can be prepared by amide formation conditions well known to skilled artisans. Compound 1 is reacted with an appropriately substituted Compound 2 or a proper salt of Compound 2 such as a hydrochloride salt, in the presence of a proper amide bond formation reagent such as HATU, 1-propanephosphonic acid cyclic anhydride, or bis(2-oxo-3-oxazolidinyl)phosphonic chloride, and a suitable base such as triethylamine in an appropriate solvent such as dimethylformamide to provide the desired compound of Formula I

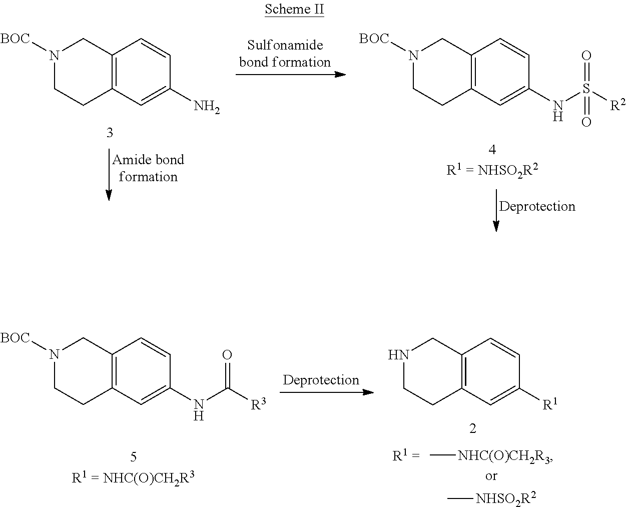

Method to make a subset of Compound 2 when R¹ is —NHSO₂R² or —NHC(O)CH₂R³

When R¹ is —NHSO₂R² or —NHC(O)CH₂R³, a subset of Compound 2 can be prepared as illustrated in Scheme II, where R² and R³ are as previously defined.

When R¹ is —NHSO₂R², Compound 3 is reacted with an appropriately substituted sulfonyl chloride, in the presence of a proper sulfonamide bond formation reagent such as HATU, 1-propanephosphonic acid cyclic anhydride, or bis(2-oxo-3-oxazolidinyl)phosphonic chloride, and a suitable base such as triethylamine in an appropriate solvent such as dimethylformamide to provide Compound 4.

When R¹ is —NHC(O)CH₂R³, Compound 3 is reacted with an appropriately substituted carboxylic acid, in the presence of a proper amide bond formation reagent such as HATU, 1-propanephosphonic acid cyclic anhydride, or bis(2-oxo-3-oxazolidinyl)phosphonic chloride, and a suitable base such as triethylamine in an appropriate solvent such as dimethylformamide to provide Compound 5.

Compound 4 or 5 can be de-protected by a suitable de-protecting reagent such as trifluoroacetic acid or hydrochloric acid to give Compound 2 when R¹ is —NHSO₂R² or —NHC(O)CH₂R³.

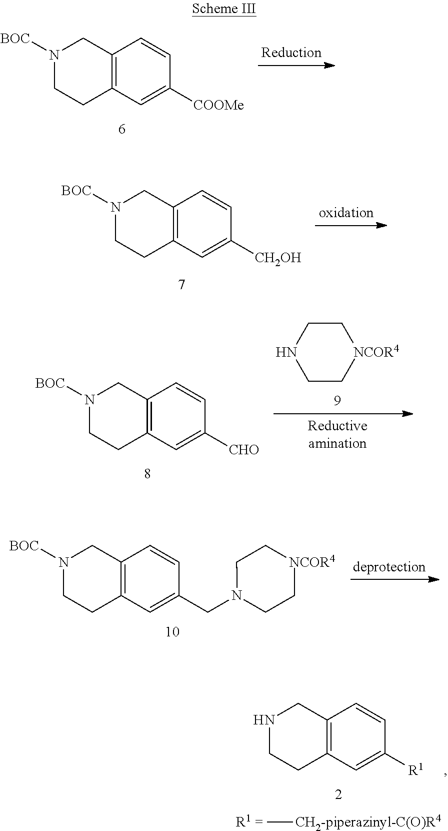

Method to make a subset of Compound 2 when R¹ is —CH₂-piperazinyl-C(O)R⁴

When R¹ is —CH₂-piperazinyl-C(O)R⁴, a subset of Compound 2 can be prepared as illustrated in Scheme III, where R⁴ is as previously defined.

Compound 6 is reduced with a proper reducing reagent such as lithium aluminum hydride in an appropriate solvent such as tetrahydrofuran to provide Compound 7. Compound 7 can be oxidized by an oxidative reagent such as manganese (IV) oxide in a suitable solvent such as dichloromethane to provide Compound 8, which is further reacted with Compound 9 under reductive amination conditions well known to a skilled artisan to provide Compound 10. For example, Compound 8 can react with Compound 9 with the existence of a suitable reducing reagent such a triacetoxyborohydride and a proper acid such as acetic acid, in a proper solvent such as dichloromethane to provide Compound 10. Compound 10 can be de-protected by a suitable de-protecting reagent such as trifluoroacetic acid or hydrochloric acid to give Compound 2 when R¹ is —CH₂— piperazinyl-C(O)R⁴.

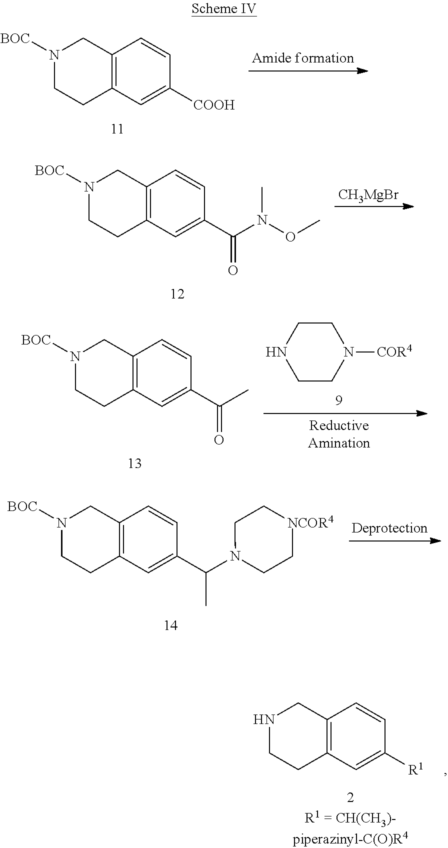

Method to make a subset of Compound 2 when R¹ is —CH(CH₃)-piperazinyl-C(O)R⁴

When R¹ is —CH(CH₃)-piperazinyl-C(O)R⁴, a subset of Compound 2 can be prepared as illustrated in Scheme IV, where R⁴ is as previously defined.

Compound 11 can react with N,O-dimethylhydroxylamine hydrochloride under amide bond formation condition described above to provide Compound 12. Compound 12 can react with methyl magnesium bromide in a proper solvent such as tetrahydrofuran to provide Compound 13. Compound 13 can reacted with Compound 9 under reductive amination conditions described above to provide Compound 14, which can be further de-protected by a suitable de-protecting reagent such as trifluoroacetic acid or hydrochloric acid to give Compound 2 when R¹ is —CH(CH₃)-piperazinyl-C(O)R⁴.

### PREPARATION 1

### tert-Butyl 6-(methanesulfonamido)-3,4-dihydro-1H-isoquinoline-2-carboxylate

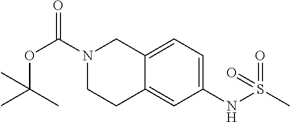 [SMILES: CC(C)(C)OC(=O)N1CCc2cc(NS(C)(=O)=O)ccc2C1]

Add methanesulfonyl chloride (62.16 g, 42.00 mL, 542.63 mmol) to a solution of tert-butyl 6-amino-3,4-dihydro-1H-isoquinoline-2-carboxylate (90.00 g, 362.43 mmol) and triethylamine (73.35 g, 101.03 mL, 724.86 mmol) in dichloromethane (900 mL), which is cooled to 0° C. Stir the mixture at room temperature overnight.

Treat the mixture with aqueous sodium hydroxide solution (2 M, 400 mL) and heat the reaction mixture to reflux for 48 hours. Cool to room temperature, add water (200 mL) and separate the phases. Extract the aqueous layer with ethyl acetate (500 mL), combine the organic layers and wash with saturated aqueous sodium chloride (250 mL) and concentrate under reduced pressure to a red-orange oil. Dissolve the red-orange oil in dichloromethane (300 mL) and heptanes (500 mL) and concentrate to dryness, and then keep the material in vacuum oven at 45° C. overnight to afford the title compound (120 g, 367.63 mmol). MS (m/z): 271 (M+1-tBu) as a light orange solid.

The following compounds are prepared essentially by the method of Preparation 1.

| Prep. | | | MS |
|---|---|---|---|
| No. | Compound Name | Structure | (m/z): |
| 2 | tert-Butyl 6-(2-pyridylsulfonylamino)-3,4- dihydro-1H-isoquinoline-2-carboxylate | 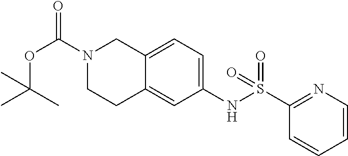 [SMILES: CC(C)(C)OC(=O)N1CCc2cc(NS(=O)(=O)c3ccccn3)ccc2C1] | 334 (M + 1− tBu) |
| 3 | tert-Butyl 6-(tetrahydropyran-4- ylsulfonylamino)-3,4-dihydro-1H- isoquinoline-2-carboxylate | 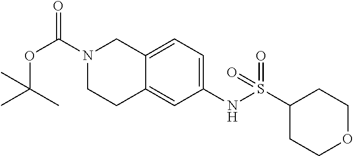 [SMILES: CC(C)(C)OC(=O)N1CCc2cc(NS(=O)(=O)C3CCOCC3)ccc2C1] | 297 (M + 1 − BOC) |
| 4 | tert-Butyl 6- (cyclopropylmethylsulfonylamino)-3,4- dihydro-1H-isoquinoline-2-carboxylate | 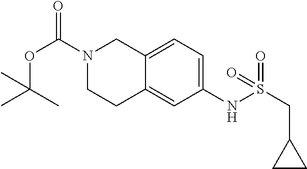 [SMILES: CC(C)(C)OC(=O)N1CCc2cc(NS(=O)(=O)CC3CC3)ccc2C1] | 267 (M + 1 − BOC) |
| 5 | tert-Butyl 6-(3,3,3- trifluoropropylsulfonylamino)-3,4-dihydro- 1H-isoquinoline-2-carboxylate | 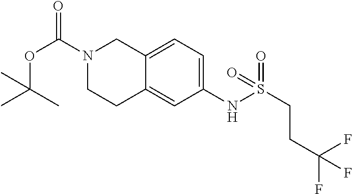 [SMILES: CC(C)(C)OC(=O)N1CCc2cc(NS(=O)(=O)CCC(F)(F)F)ccc2C1] | 353 (M + 1 − tBu) |
| 6 | tert-Butyl 6-(vinylsulfonylamino)-3,4- dihydro-1H-isoquinoline-2-carboxylate | 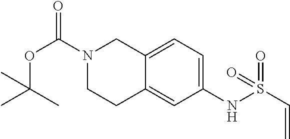 [SMILES: C=CS(=O)(=O)Nc1ccc2c(c1)CCN(C(=O)OC(C)(C)C)C2] | 239 (M + 1 − BOC) |

### PREPARATION 7

### tert-Butyl 6-(2-methoxyethylsulfonylamino)-3,4-dihydro-1H-isoquinoline-2-carboxylate

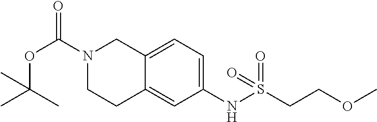 [SMILES: COCCS(=O)(=O)Nc1ccc2c(c1)CCN(C(=O)OC(C)(C)C)C2]

Add triethylamine (42.71 g, 58.82 mL, 422.03 mmol) to a solution of tert-butyl 6-amino-3,4-dihydro-1H-isoquinoline-2-carboxylate (52.40 g, 211.01 mmol) in dry tetrahydrofuran (500 mL) at 0° C., followed by 2-methoxyethanesulfonyl chloride (42.71 g, 58.82 mL, 422.03 mmol) over 25 minutes. Stir the reaction at room temperature for 90 minutes. Cool the reaction at 0° C. and add water (250 mL) over 5 minutes. Extract with methyl tert butyl ether. Wash the organic layer with water (2×250 mL) and saturated aqueous sodium chloride (250 mL). Dry the organic layer over magnesium sulfate, filter and concentrate under reduced pressure to afford the title compound (65 g, 211.87 mmol). MS (m/z): 271 (M+1-BOC).

### PREPARATION 8

### tert-Butyl 6-[(2-hydroxy-2-methyl-propyl)sulfonylamino]-3,4-dihydro-1H-isoquinoline-2-carboxylate

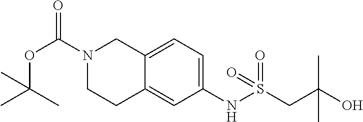 [SMILES: CC(C)(O)CS(=O)(=O)Nc1ccc2c(c1)CCN(C(=O)OC(C)(C)C)C2]

Add slowly n-butyl lithium 2.5 M in hexane (255.30 g, 370.00 mL, 925.00 mmol) to a solution of tert-butyl 6-(methanesulfonamido)-3,4-dihydro-1H-isoquinoline-2-carboxylate (120.00 g, 367.63 mmol) in tetrahydrofuran (2 L) at −78° C. Stir the mixture at −78° C. for 30 minutes and add acetone (27.65 g, 35.00 mL, 476.13 mmol) to the mixture over a 5 minute period and allow it to warm to room temperature. Add saturated aqueous sodium bicarbonate solution (200 mL) to the mixture, dilute with water (400 mL) and then separate the phases. Extract the aqueous layer with ethyl acetate (4×800 mL), combine the organic layers, dry over anhydrous sodium sulfate, filter and concentrate under reduced pressure. Purify by silica gel chromatography, eluting with a mobile phase of hexane/ethyl acetate (0% to 50% ethyl acetate over 90 minutes), to afford the title compound (48.00 g, 124.84 mmol). MS (m/z): 285 (M+1-BOC). Combine fractions containing a mixture of peaks from the previous purification to give 20 g of material. Purify it by silica gel chromatography, eluting with a mobile phase of chloroform/isopropanol (0% to 5% isopropanol over 45 minutes), to afford the additional title compound (5.60 g, 14.56 mmol). MS (m/z): 285 (M+1-BOC).

### PREPARATION 9

### tert-Butyl 6-(2-isopropoxyethylsulfonylamino)-3,4-dihydro-1H-isoquinoline-2-carboxylate

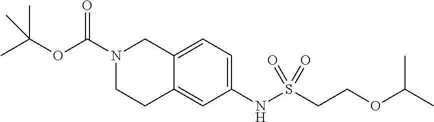 [SMILES: CC(C)OCCS(=O)(=O)Nc1ccc2c(c1)CCN(C(=O)OC(C)(C)C)C2]

Add to a microwave vial tert-butyl 6-(vinylsulfonylamino)-3,4-dihydro-1H-isoquinoline-2-carboxylate (635.00 mg, 1.88 mmol), sodium isopropoxide (924.03 mg, 11.26 mmol) and dry isopropyl alcohol (4.99 g, 0.6.35 mL). Heat the mixture at 110° C. for 1 hour in a microwave reactor. Pour the mixture into saturated aqueous ammonium chloride solution and extract with ethyl acetate (2×40 mL), wash the organic layer with saturated aqueous sodium chloride, dry over sodium, filter and concentrate under reduced pressure to afford the title compound (695.00 mg, 1.74 mmol). MS (m/z): 399 (M+1).

### PREPARATION 10

### tert-Butyl 6-[(3-hydroxy-3-methyl-pentanoyl)amino]-3,4-dihydro-1H-isoquinoline-2-carboxylate

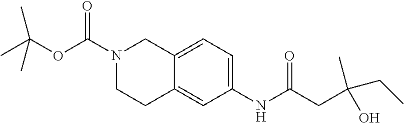 [SMILES: CCC(C)(O)CC(=O)Nc1ccc2c(c1)CCN(C(=O)OC(C)(C)C)C2]

Add triethylamine (948.3 mg, 1.3 mL, 9.3 mmol) to a solution of tert-butyl 6-amino-3,4-dihydro-1H-isoquinoline-2-carboxylate (4.7 mmoles; 1.2 g), 3-hydroxy-3-methyl-pentanoic acid (4.7 mmoles; 615.0 mg), bis(2-oxo-3-oxazolidinyl)phosphonic chloride (1.4 g, 5.6 mmol) in dichloromethane (23.3 mL). Stir the mixture at room temperature overnight. Add dichloromethane (30 mL), wash with water, dry over sodium sulfate and filter. Concentrate the filtrate under reduced pressure and dissolve the resulting oil in methanol (5 mL). Transfer the methanol solution to an ion exchange chromatography, eluting with 10% methanol/dichloromethane followed by 2 N NH₃ in methanol. Concentrate the methanol fraction to afford the title compound (1.6 g, 4.41 mmol). MS (m/z): 363 (M+1).

### PREPARATION 11

### tert-Butyl 6-[(4,4,4-trifluoro-3-hydroxy-3-methyl-butanoyl)amino]-3,4-dihydro-1H-isoquinoline-2-carboxylate

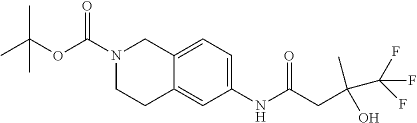 [SMILES: CC(C)(C)OC(=O)N1CCc2cc(NC(=O)CC(C)(O)C(F)(F)F)ccc2C1]

Add triethylamine (407.50 mg, 561.29 μL, 4.03 mmol) to a solution of tert-butyl 6-amino-3,4-dihydro-1H-isoquinoline-2-carboxylate (500.00 mg, 2.01 mmol), 4,4,4-trifluoro-3-hydroxy-3-methyl-butanoic acid (346.53 mg, 2.01 mmol) and (O-(7-azabenzotriazol-1-yl)-N,N,N′,N′-tetramethyluronium hexafluorophosphate (1.53 g, 4.03 mmol) in dimethylformamide (4 mL). Stir the mixture at room temperature overnight. Remove the solvent under reduced pressure, add dichloromethane (30 mL) and wash with 20 mL of water. Dry over sodium sulfate, filter and concentrate under reduced pressure to afford the title compound (0.81 g, 2.0 mmol). MS (m/z): 403 (M+1).

The following compounds are prepared essentially by the method of Preparation 11.

| Prep. | | | MS |
|---|---|---|---|
| No. | Compound Name | Structure | (m/z): |
| 12 | tert-Butyl 6-[(3- hydroxy-3-methyl- butanoyl)amino]-3,4- dihydro-1H- isoquinoline-2- carboxylate | 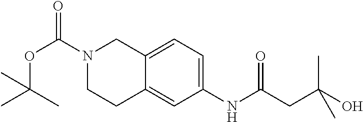 [SMILES: CC(C)(O)CC(=O)Nc1ccc2c(c1)CCN(C(=O)OC(C)(C)C)C2] | 349 (M + 1) |
| 13 | tert-Butyl 6-(3,3- dimethylbutanoylamino)- 3,4-dihydro-1H- isoquinoline-2- carboxylate | 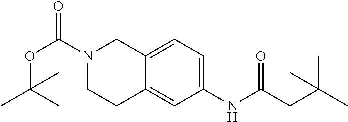 [SMILES: CC(C)(C)CC(=O)Nc1ccc2c(c1)CCN(C(=O)OC(C)(C)C)C2] | 347 (M + 1) |
| 14 | tert-Butyl 6-[(2- tetrahydropyran-2- ylacetyl)amino]-3,4- dihydro-1H- isoquinoline-2- carboxylate | 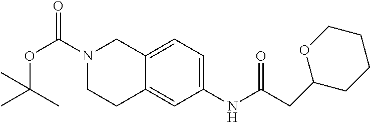 [SMILES: CC(C)(C)OC(=O)N1CCc2cc(NC(=O)CC3CCCCO3)ccc2C1] | 375 (M + 1) |

### PREPARATION 15

### 2-Hydroxy-2-methyl-N-(1,2,3,4-tetrahydroisoquinolin-6-yl)propane-1-sulfonamide hydrochloride

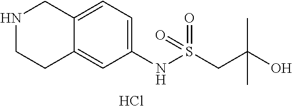 [SMILES: CC(C)(O)CS(=O)(=O)Nc1ccc2c(c1)CCNC2 ; Cl]

Add hydrogen chloride (4 M in dioxane, 120.00 g, 120.00 mL, 480.00 mmol) to a solution of tert-butyl 6-[(2-hydroxy-2-methyl-propyl)sulfonylamino]-3,4-dihydro-1H-isoquinoline-2-carboxylate (53.6 g, 139.22 mmol) in 1,4-dioxane (400 mL) and stir the mixture at room temperature overnight. Concentrate the mixture under reduced pressure and further dry it under vacuum at 50° C. to afford the title compound (46.90 g, 146.18 mmol). MS (m/z): 285 (M+1-HCl).

The following compounds are prepared essentially by the method of Preparation 15.

| Prep. | | | MS |
|---|---|---|---|
| No. | Compound Name | Structure | (m/z): |
| 16 | N-(1,2,3,4- Tetrahydroisoquinolin- 6-yl)pyridine-2- sulfonamide hydrochloride | 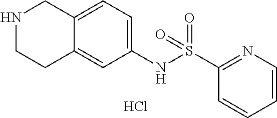 [SMILES: O=S(=O)(Nc1ccc2c(c1)CCNC2)c1ccccn1 ; Cl] | 290 (M + 1 − HCl) |
| 17 | N-(1,2,3,4- Tetrahydroisoquinolin- 6-yl)tetrahydropyran- 4-sulfonamide hydrochloride | 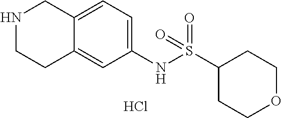 [SMILES: O=S(=O)(Nc1ccc2c(c1)CCNC2)C1CCOCC1 ; Cl] | 297 (M + 1 − HCl) |
| 18 | 3,3,3-Trifluoro-N- (1,2,3,4- tetrahydroisoquinolin- 6-yl)propane-1- sulfonamide hydrochloride | 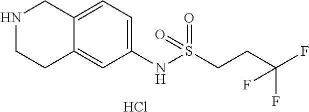 [SMILES: O=S(=O)(CCC(F)(F)F)Nc1ccc2c(c1)CCNC2 ; Cl] | 309 (M + 1 − HCl) |
| 19 | 1-Cyclopropyl-N- (1,2,3,4- tetrahydroisoquinolin- 6- yl)methanesulfonamide hydrochloride | 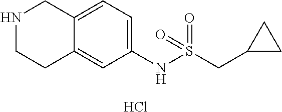 [SMILES: O=S(=O)(CC1CC1)Nc1ccc2c(c1)CCNC2 ; Cl] | 267 (M + 1 − HCl) |
| 20 | 2-Isopropoxy-N- (1,2,3,4- tetrahydroisoquinolin-6- yl)ethanesulfonamide hydrochloride | 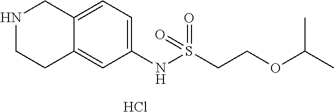 [SMILES: CC(C)OCCS(=O)(=O)Nc1ccc2c(c1)CCNC2 ; Cl] | 299 (M + 1 − HCl) |
| 21 | 3-Hydroxy-3-methyl- N-(1,2,3,4- tetrahydroisoquinolin- 6-yl)pentanamide hydrochloride | 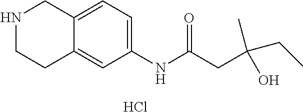 | 299 (M + 1 − HCl) |
| 22 | 4,4,4-Trifluoro-3- hydroxy-3-methyl-N- (1,2,3,4- tetrahydroisoquinolin- 6-yl)butanamide hydrochloride | 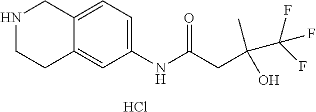 [SMILES: CC(O)(CC(=O)Nc1ccc2c(c1)CCNC2)C(F)(F)F ; Cl] | 303 (M + 1 − HCl) |
| 23 | N-(1,2,3,4- Tetrahydroisoquinolin- 6-yl)-2- tetrahydropyran-2-yl- acetamide hydrochloride | 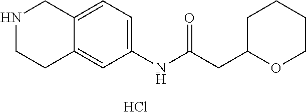 [SMILES: O=C(CC1CCCCO1)Nc1ccc2c(c1)CCNC2 ; Cl] | 275 (M + 1 − HCl) |
| 24 | 3,3-Dimethyl-N- (1,2,3,4- tetrahydroisoquinolin- 6-yl)butanamide hydrochloride | 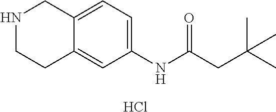 [SMILES: CC(C)(C)CC(=O)Nc1ccc2c(c1)CCNC2 ; Cl] | 247 (M + 1 − HCl) |
| 25 | 3-Hydroxy-3-methyl- N-(1,2,3,4- tetrahydroisoquinolin- 6-yl)butanamide hydrochloride | 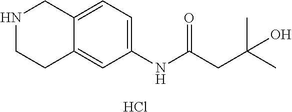 [SMILES: CC(C)(O)CC(=O)Nc1ccc2c(c1)CCNC2 ; Cl] | 249 (M + 1 − HCl) |

### PREPARATION 26

### 2-Methoxy-N-(1,2,3,4-tetrahydroisoquinolin-6-yl)ethanesulfonamide hydrochloride

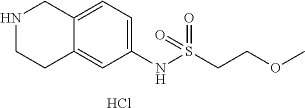 [SMILES: COCCS(=O)(=O)Nc1ccc2c(c1)CCNC2 ; Cl]

Add acetyl chloride (60.31 mL, 66.53 g, 847.49 mmol) over 15 minutes to isopropyl alcohol (235.83 g, 300.00 mL, 3.92 mol) at 0° C., stir the solution at room temperature for 30 minutes. Add tert-butyl 6-(2-methoxyethylsulfonylamino)-3,4-dihydro-1H-isoquinoline-2-carboxylate (84.40 g, 211.87 mmol) in isopropyl alcohol (78.61 g, 100.00 mL, 1.31 mol) and heat the reaction mixture at 40° C. for 90 minutes. Cool the mixture to room temperature and later at 4° C. for 30 minutes. Filter the solid to afford the title compound (58.80 g, 191.65 mmol). MS (m/z): 271 (M+1).

### PREPARATION 27

### 1,2,3,4-Tetrahydroisoquinolin-6-amine

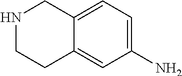 [SMILES: Nc1ccc2c(c1)CCNC2]

Add hydrogen chloride (4 M in dioxane, 32.22 mL, 128.86 mmol) dropwise to a solution of tert-butyl 6-amino-3,4-dihydro-1H-isoquinoline-2-carboxylate (8.00 g, 32.22 mmol) in dichloromethane (120 mL) at room temperature. Stir the mixture for 1.5 hour. Concentrate under reduced pressure and load onto an SCX-2 cartridge (100 g), wash with methanol and elute with 2 M methanolic ammonia. Concentrate the methanol washings under reduced pressure and reload onto the SCX-2 cartridge (50 g), wash with methanol and elute with 2 M methanolic ammonia. Combine and concentrate the basic eluents from both purifications to afford the title compound (4.85 g, 32.72 mmol). MS (m/z): 149 (M+1).

### PREPARATION 28

### 1-(6-Amino-3,4-dihydro-1H-isoquinolin-2-yl)-2-(3-pyridyloxy)ethanone

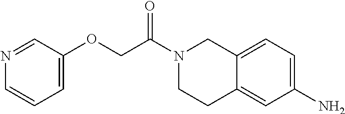 [SMILES: Nc1ccc2c(c1)CCN(C(=O)COc1cccnc1)C2]

Add 2-(3-pyridyloxy)acetic acid (5.27 g, 34.41 mmol) and 1,1′-carbonyldiimidazole (5.58 g, 34.41 mmol) to a flask and purge with N₂, then add dry tetrahydrofuran (102.00 mL) and heat to 45° C. for 1 hour. Transfer the mixture to a dropping funnel and add the mixture over 1 hour to a N₂ purged flask containing a suspension of 1,2,3,4-tetrahydroisoquinolin-6-amine (5.10 g, 34.41 mmol) in dimethylformamide (40.80 mL). Stir the mixture for 1 hour and concentrate under reduced pressure. Add tetrahydrofuran (30 mL) and stir until a precipitate forms. Cool the mixture in ice bath and filter, and then wash the residue with tetrahydrofuran (10 mL). Dry the solid to afford the title compound (5.66 g, 19.98 mmol). MS (m/z): 284 (M+1). Concentrate the liquors under reduced pressure and purify by flash chromatography (silica gel: 80 g; load with dichloromethane (40 mL); isocratic flow: 5% methanol in dichloromethane). to afford the title compound (3.21 g, 1.33 mmol). MS (m/z): 284 (M+1).

### PREPARATION 29

### tert-Butyl 4-[[2-[2-(3-pyridyloxy)acetyl]-3,4-dihydro-1H-isoquinolin-6-yl]sulfamoyl]piperidine-1-carboxylate

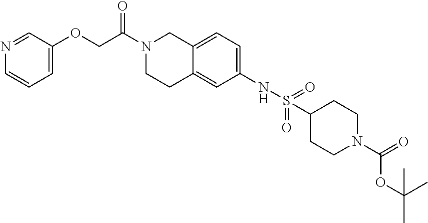 [SMILES: CC(C)(C)OC(=O)N1CCC(S(=O)(=O)Nc2ccc3c(c2)CCN(C(=O)COc2cccnc2)C3)CC1]

Add tert-butyl 4-chlorosulfonylpiperidine-1-carboxylate (1.00 g, 676.74 μL, 3.53 mmol) to a suspension of 1-(6-amino-3,4-dihydro-1H-isoquinolin-2-yl)-2-(3-pyridyloxy)ethanone (1.00 g, 3.53 mmol) in dichloromethane (17.65 mL). Then add triethylamine (1.48 mL, 10.59 mmol) and stir at room temperature for 2 hours. Add tert-butyl 4-chlorosulfonylpiperidine-1-carboxylate (500.79 mg, 338.37 μL, 1.76 mmol) and stir for 1 hour at room temperature. Add tert-butyl 4-chlorosulfonylpiperidine-1-carboxylate (200.31 mg, 135.35 μL, 705.89 μmol) and stir at room temperature for 30 minutes. Add dichloromethane (50 mL) and wash with saturated aqueous sodium chloride (50 mL). Dry the organic phase over sodium sulfate, filter and concentrate under reduced pressure. Purify by flash chromatography with acetone to afford the title compound (1.47 g, 2.63 mmol). MS (m/z): 531 (M+1).

### PREPARATION 30

### N-[2-[2-(3-Pyridyloxy)acetyl]-3,4-dihydro-1H-isoquinolin-6-yl]piperidine-4-sulfonamide

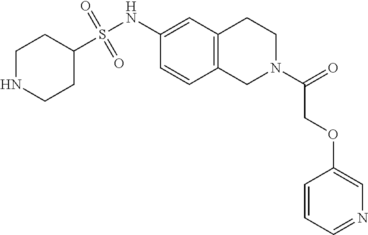 [SMILES: O=C(COc1cccnc1)N1CCc2cc(NS(=O)(=O)C3CCNCC3)ccc2C1]

Add hydrogen chloride (4.14 g, 3.95 mL, 15.79 mmol) to a solution of tert-butyl 4-[[2-[2-(3-pyridyloxy)acetyl]-3,4-dihydro-1H-isoquinolin-6-yl]sulfamoyl]piperidine-1-carboxylate (1.47 g, 2.63 mmol) in dichloromethane (13.16 mL) and stir at room temperature for 1 hour. Add hydrogen chloride (4.14 g, 3.94 mL, 15.8 mmol) and stir at room temperature for 16 hours. Concentrate under reduced pressure. Load onto an SCX-2 cartridge, wash with methanol and elute with

2 M methanolic ammonia. Concentrate under reduced pressure the basic fraction to afford the title compound (1.34 g, 2.43 mmol). MS (m/z): 431 (M+1).

### PREPARATION 31

### 6-Hydroxymethyl-3,4-dihydro-1H-isoquinoline-2-carboxylic acid tert-butyl ester

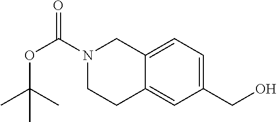 [SMILES: CC(C)(C)OC(=O)N1CCc2cc(CO)ccc2C1]

Add lithium aluminum hydride (716.49 mg, 18.88 mmol) dropwise to a solution of O2-tert-butyl 06-methyl 3,4-dihydro-1H-isoquinoline-2,6-dicarboxylate (5.00 g, 17.16 mmol) in tetrahydrofuran (85.81 mL) at 0° C. and stir at that temperature for 1 hour. Then add water (6 mL) and stir for 15 minutes at 0° C., filter over CELITE® and wash the CELITE® with ethyl acetate. Concentrate the filtrate under reduced pressure to afford the title compound (4.5 g, 17.09 mmol). MS (m/z): 264 (M+1).

### PREPARATION 32

### tert-Butyl 6-formyl-3,4-dihydro-1H-isoquinoline-2-carboxylate

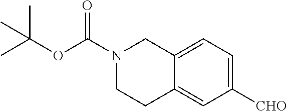 [SMILES: CC(C)(C)OC(=O)N1CCc2cc(C=O)ccc2C1]

Add activated manganese(IV) oxide (2.85 g, 32.77 mmol) to a solution of 6-hydroxymethyl-3,4-dihydro-1H-isoquinoline-2-carboxylic acid tert-butyl ester (863.00 mg, 3.28 mmol) in dichloromethane (50.00 mL) at room temperature and stir at that temperature for 16 hours. Filter over a pad of CELITE® and wash the CELITE® pad with dichloromethane. Concentrate the filtrate under reduced pressure to afford the title compound (740 mg, 2.83 mmol). MS (m/z): 206 (M+1-tBu).

### PREPARATION 33

### tert-Butyl 6-[methoxy(methyl)carbamoyl]-3,4-dihydro-1H-isoquinoline-2-carboxylate

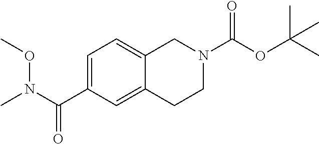 [SMILES: CON(C)C(=O)c1ccc2c(c1)CCN(C(=O)OC(C)(C)C)C2]

Add 4-(4,6-dimethoxy-1,3,5-triazin-2-yl)-4-methylmorpholinium chloride (26.34 g, 94.86 mmol) to solution of 2-tert-butoxycarbonyl-3,4-dihydro-1H-isoquinoline-6-carboxylic acid (18.79 g, 67.76 mmol), N,O-dimethylhydroxylamine hydrochloride (7.93 g, 81.31 mmol) and N-methylmorpholine (13.71 g, 14.95 mL, 135.51 mmol) in methanol (563.70 mL). Stir the mixture at room temperature overnight. Concentrate under reduced pressure. Then add ethyl acetate (250 mL) and water (250 mL) to the mixture. Separate the layers and extract the aqueous layer with ethyl acetate (150 mL). Combine the organic layers, wash with aqueous hydrochloric acid solution (2 M, 200 mL), wash with saturated aqueous sodium chloride (200 mL) and dry over sodium sulfate. Concentrate under reduced pressure to afford the title compound (24.28 g, 75.78 mmol). MS (m/z): 321 (M+1).

### PREPARATION 34

### tert-Butyl 6-acetyl-3,4-dihydro-1H-isoquinoline-2-carboxylate

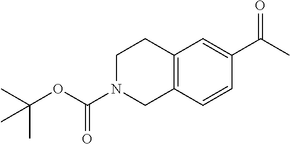 [SMILES: CC(=O)c1ccc2c(c1)CCN(C(=O)OC(C)(C)C)C2]

Add methyl magnesium bromide (78.44 g, 75.78 mL, 227.35 mmol) dropwise over 1 hour to a solution of tert-butyl 6-[methoxy(methyl)carbamoyl]-3,4-dihydro-1H-isoquinoline-2-carboxylate (24.28 g, 75.78 mmol) in tetrahydrofuran (505 mL) under N₂ and at −5° C. Purge the flask with N₂ and charge with dry tetrahydrofuran (505.22 mL). Stir the mixture under the addition for 50 minutes at −5° C. Pour onto an saturated aqueous ammonium chloride solution, stir 5 minutes and dilute with methyl tert butyl ether (150 mL). Separate the layer, wash the organic layer with saturated aqueous sodium chloride and dry over magnesium sulfate. Concentrate the filtrate under reduced pressure to afford the title compound (20.4 g, 74.09 mmol). MS (m/z): 276 (M+1).

### PREPARATION 35

### Piperazin-1-yl(tetrahydropyran-4-yl)methanone

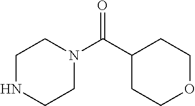 [SMILES: O=C(C1CCOCC1)N1CCNCC1]

Add tetrahydropyran-4-carbonyl chloride (1.2 g, 8.1 mmol) to a solution of tert-butyl piperazine-1-carboxylate (1.5 g, 8.1 mmol) in dichloromethane (30 mL). Then add triethylamine (896.4 mg, 1.2 mL, 8.9 mmol) and stir at room temperature for 2 hours. Add water (30 mL), extract with dichloromethane (30 mL), dry over sodium sulfate and concentrate under reduced pressure. Add dichloromethane (30 mL) to the obtained material and add hydrogen chloride (10.6 g, 10.1 mL, 40.3 mmol) and stir at room temperature for 30 minutes. Concentrate under reduced pressure, load onto an SCX-2 cartridge, wash with methanol and elute with 2 M methanolic ammonia. Concentrate the basic fraction under reduced pressure to afford the title compound (1.6 g, 8.1 mmol). MS (m/z): 199 (M+1).

### PREPARATION 36

### 2-Morpholino-1-piperazin-1-yl-ethanone

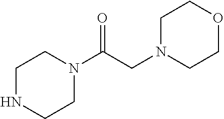 [SMILES: O=C(CN1CCOCC1)N1CCNCC1]

Add triethylamine (1.36 g, 1.87 mL, 13.42 mmol) to a solution of tert-butyl piperazine-1-carboxylate (1.00 g, 5.37 mmole), 2-morpholinoacetic acid (779.36 mg, 5.37 mmol) in dimethylformamide (26.85 mL), O-(7-azabenzotriazol-1-yl)-N,N,N′,N′-tetramethyluronium hexafluorophosphate (2.59 g, 8.05 mmol) and stir at room temperature for 4 hours. Concentrate under reduced pressure and dissolve in dichloromethane (30 mL) and add hydrogen chloride (7.05 g, 6.71 mL, 26.85 mmol). Stir the mixture at room temperature for 30 minutes. Concentrate under reduced pressure, load onto an SCX-2 cartridge, wash with methanol and elute with 2 M methanolic ammonia. Concentrate the basic fraction under reduced pressure to afford the title compound (1.4 g, 6.58 mmol). MS (m/z): 214 (M+1).

The following compounds are prepared essentially by the method of Preparation 36.

| Prep. | | | MS |
|---|---|---|---|
| No. | Compound Name | Structure | (m/z): |
| 37 | 1-Piperazin-1- yl-2- tetrahydropyran- 4-yl- ethanone | 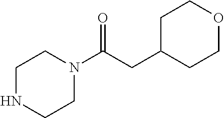 [SMILES: O=C(CC1CCOCC1)N1CCNCC1] | 213 (M + 1) |
| 38 | 3-Hydroxy- 3-methyl-1- piperazin-1- yl-butan-1- one | 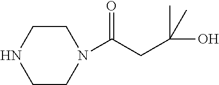 | 187 (M + 1) |

### PREPARATION 39

### tert-Butyl 6-[[4-(tetrahydropyran-4-carbonyl)piperazin-1-yl]methyl]-3,4-dihydro-1H-isoquinoline-2-carboxylate

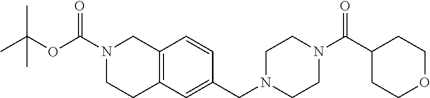 [SMILES: CC(C)(C)OC(=O)N1CCc2cc(CN3CCN(C(=O)C4CCOCC4)CC3)ccc2C1]

Add acetic acid (104.80 mg, 100.00 μL, 1.75 mmol) to a solution of piperazin-1-yl(tetrahydropyran-4-yl)methanone (796.64 mg, 4.02 mmol), tert-butyl 6-formyl-3,4-dihydro-1H-isoquinoline-2-carboxylate (700.00 mg, 2.68 mmol) in dichloromethane (13.39 mL). Stir 15 minutes at room temperature and add sodium triacetoxyborohydride (681.28 mg, 3.21 mmol) and stir at room temperature overnight. Concentrate under reduced pressure, load onto an SCX-2 cartridge, wash with methanol and elute with 2 M methanolic ammonia. Concentrate the basic fraction under reduced pressure. Then purify by flash chromatography with hexane:ethyl acetate (1:1) to ethyl acetate:methanol (95:5) to afford the title compound (0.71 g, 1.6 mmol). MS (m/z): 444 (M+1).

The following compounds are prepared essentially by the method of Preparation 39.

| Prep. | | | MS |
|---|---|---|---|
| No. | Compound Name | Structure | (m/z): |
| 40 | tert-Butyl 6-[[4-(2- morpholinoacetyl) piperazin-1-yl]methyl]- 3,4-dihydro-1H- isoquinoline-2- carboxylate | 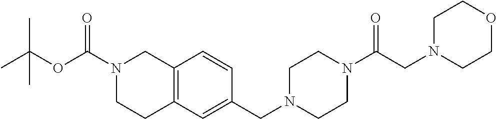 [SMILES: CC(C)(C)OC(=O)N1CCc2cc(CN3CCN(C(=O)CN4CCOCC4)CC3)ccc2C1] | 459 (M+) |
| 41 | tert-Butyl 6-[[4-(2- tetrahydropyran-4- ylacetyl)piperazin-1- yl]methyl]-3,4- dihydro-1H-isoquinoline- 2-carboxylate | 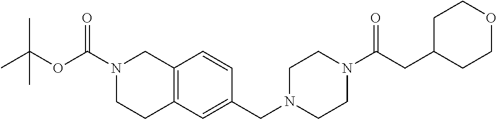 [SMILES: CC(C)(C)OC(=O)N1CCc2cc(CN3CCN(C(=O)CC4CCOCC4)CC3)ccc2C1] | 458 (M + 1) |

### PREPARATION 42

### tert-Butyl 6-[[4-(3-hydroxy-3-methyl-butanoyl)piperazin-1-yl]methyl]-3,4-dihydro-1H-isoquinoline-2-carboxylate

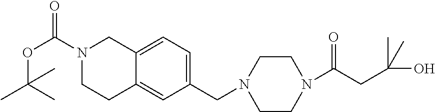 [SMILES: CC(C)(O)CC(=O)N1CCN(Cc2ccc3c(c2)CCN(C(=O)OC(C)(C)C)C3)CC1]

Add titanium tetra(isopropoxide) (818.47 mg, 853.20 μL, 2.88 mmol) to a solution of tert-butyl 6-formyl-3,4-dihydro-1H-isoquinoline-2-carboxylate (602.00 mg, 2.30 mmol) and 3-hydroxy-3-methyl-1-piperazin-1-yl-butan-1-one (557.79 mg, 2.99 mmol) in ethanol (3.8 mL). Stir the mixture at 60° C. overnight. Cool to room temperature and add to the mixture sodium tetrahydroborate (130.73 mg, 3.46 mmol) and stir overnight. Then add water and stir for 48 hours, filter over a frit and later over CELITE®. Concentrate under reduced pressure, load onto an SCX-2 cartridge, wash with methanol and elute with 2 M methanolic ammonia. Concentrate the basic fraction under reduced pressure to afford the title compound (0.035 g, 0.08 mmol). MS (m/z): 432 (M+1).

### PREPARATION 43

### tert-Butyl 6-[1-[4-(tetrahydropyran-4-carbonyl)piperazin-1-yl]ethyl]-3,4-dihydro-1H-isoquinoline-2-carboxylate

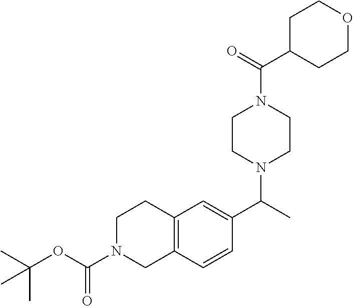 [SMILES: CC(c1ccc2c(c1)CCN(C(=O)OC(C)(C)C)C2)N1CCN(C(=O)C2CCOCC2)CC1]

Add titanium tetra(isopropoxide) (2.52 g, 2.63 mL, 8.86 mmol) to a solution of tert-butyl 6-acetyl-3,4-dihydro-1H-isoquinoline-2-carboxylate (1.22 g, 4.43 mmol) and piperazin-1-yl(tetrahydropyran-4-yl)methanone (1.05 g, 5.32 mmol) in tetrahydrofuran (4.03 mL) under N₂. Stir the mixture at 50° C. for 20 hours. Cool the mixture in an ice bath and add sodium tetrahydroborate (502.88 mg, 13.29 mmol). Stir the mixture at room temperature for 30 minutes. Cool in an ice bath and quench dropwise with a 50% aqueous citric acid solution (20 mL) and stir for 30 minutes. Separate the phases, extract the organic layer with a 50% saturated aqueous citric acid solution (10 mL). Combine the aqueous layers and neutralize with an aqueous potassium carbonate solution until pH 10. Extract with ethyl acetate (2×20 mL), wash the organic layers with water (15 mL) and saturated aqueous sodium chloride (15 mL), dry the organic phase over magnesium sulfate, filter and concentrate under reduced pressure to afford the title compound (1.2 g, 2.62 mmol). MS (m/z): 458 (M+1).

### PREPARATION 44

### [4-(1,2,3,4-Tetrahydroisoquinolin-6-ylmethyl)piperazin-1-yl]-tetrahydropyran-4-yl-methanone

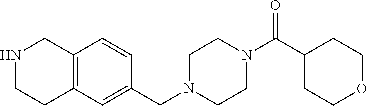 [SMILES: O=C(C1CCOCC1)N1CCN(Cc2ccc3c(c2)CCNC3)CC1]

Add trifluoroacetic acid (547.52 mg, 363.07 μL, 4.80 mmol) to a solution of tert-butyl 6-[[4-(tetrahydropyran-4-carbonyl)piperazin-1-yl]methyl]-3,4-dihydro-1H-isoquinoline-2-carboxylate (710.00 mg, 1.60 mmol) in dichloromethane (8.00 mL) and stir at room temperature for 15 minutes. Concentrate under reduced pressure, load onto an SCX-2 cartridge, wash with methanol and elute with 2 M methanolic ammonia. Concentrate the basic fraction under reduced pressure to afford the title compound (0.490 g, 1.43 mmol). MS (m/z): 344 (M+1).

### PREPARATION 45

### [4-[1-(1,2,3,4-Tetrahydroisoquinolin-6-yl)ethyl]piperazin-1-yl]-tetrahydropyran-4-yl-methanone

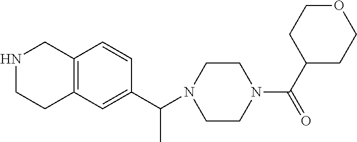 [SMILES: CC(c1ccc2c(c1)CCNC2)N1CCN(C(=O)C2CCOCC2)CC1]

Preparation 45 is prepared essentially by the method of Preparation 44. MS (m/z): 358 (M+1).

### PREPARATION 46

### 2-Morpholino-1-[4-(1,2,3,4-tetrahydroisoquinolin-6-ylmethyl)piperazin-1-yl]ethanone

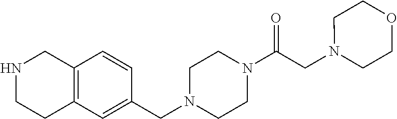 [SMILES: O=C(CN1CCOCC1)N1CCN(Cc2ccc3c(c2)CCNC3)CC1]

Add hydrogen chloride (206.06 mg, 196.25 μL, 785.00 μmol) to a solution of tert-butyl 6-[[4-(2-morpholinoacetyl)piperazin-1-yl]methyl]-3,4-dihydro-1H-isoquinoline-2-carboxylate (120.00 mg, 261.67 μmol) in dichloromethane (5.00 mL) and stir for 10 minutes at room temperature. Concentrate under reduced pressure, load onto an SCX-2 cartridge, wash with methanol and elute with 2 M methanolic ammonia. Concentrate the basic fraction under reduced pressure to afford the title compound (0.085 g, 0.24 mmol). MS (m/z): 359 (M+1).

The following compounds are prepared essentially by the method of Preparation 46.

| Prep. | | | MS |
|---|---|---|---|
| No. | Compound Name | Structure | (m/z): |
| 47 | 1-[4-(1,2,3,4- Tetrahydroisoquinolin- 6-ylmethyl)piperazin-1- yl]-2-tetrahydropyran-4- yl-ethanone | 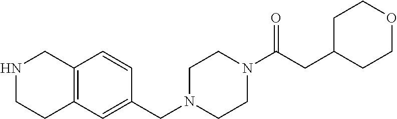 [SMILES: O=C(CC1CCOCC1)N1CCN(Cc2ccc3c(c2)CCNC3)CC1] | 358 (M + 1) |
| 48 | 3-Hydroxy-3-methyl-1- [4-(1,2,3,4- tetrahydroisoquinolin-6- ylmethyl)piperazin-1- yl]butan-1-one | 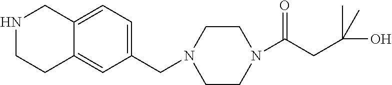 [SMILES: CC(C)(O)CC(=O)N1CCN(Cc2ccc3c(c2)CCNC3)CC1] | 332 (M + 1) |

### Example 1

### 2-Hydroxy-2-methyl-N-[2-[2-(3-pyridyloxy)acetyl]-3,4-dihydro-1H-isoquinolin-6-yl]propane-1-sulfonamide

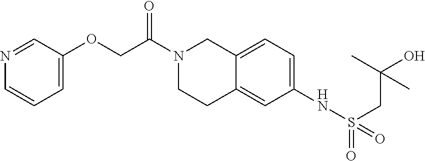 [SMILES: CC(C)(O)CS(=O)(=O)Nc1ccc2c(c1)CCN(C(=O)COc1cccnc1)C2]

Add slowly 1-propanephosphonic acid cyclic anhydride (122.30 g, 100.00 mL, 192.18 mmol) to a solution of 2-hydroxy-2-methyl-N-(1,2,3,4-tetrahydroisoquinolin-6-yl)propane-1-sulfonamide hydrochloride (46.90 g, 146.18 mmol), 2-(3-pyridyloxy)acetic acid (27.00 g, 176.31 mmol), dimethylformamide (708.98 g, 750.00 mL, 9.70 mol), triethylamine (59.53 g, 82.00 mL, 588.31 mmol) at 0° C. Allow the reaction to warm slowly to room temperature and stir overnight. Add saturated aqueous sodium sulfate solution (500 mL) and water (500 mL). Extract with dichloromethane (3×1 L), combine the organic layers, dry over anhydrous sodium sulfate, filter and concentrate under reduced pressure. Purify by silica gel chromatography eluting with a mobile phase of dichloromethane/methanol (0% to 10% methanol over 90 minutes). Combine fractions containing a mixture of peaks from previous purification and purify by silica gel chromatography eluting with a mobile phase of dichloromethane/methanol (0% to 10% methanol over 45 minutes) to afford the title compound (8.00 g, 19.05 mmol).

Combine the two fractions to afford the title compound (38.00 g, 90.58 mmol). MS (m/z): 420 (M+1).

### Example 2

### 2-Methoxy-N-[2-[2-(3-pyridyloxy)acetyl]-3,4-dihydro-1H-isoquinolin-6-yl]ethanesulfonamide

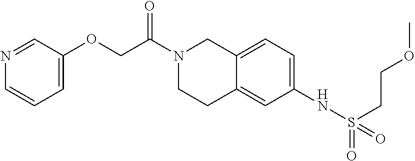 [SMILES: COCCS(=O)(=O)Nc1ccc2c(c1)CCN(C(=O)COc1cccnc1)C2]

Add triethylamine (76.12 g, 104.85 mL, 752.25 mmol) over a minute to a suspension of 2-(3-pyridyloxy)acetic acid (33.12 g, 216.27 mmol) and 2-methoxy-N-(1,2,3,4-tetrahydroisoquinolin-6-yl)ethanesulfonamide hydrochloride (57.70 g, 188.06 mmol) in ethyl acetate (1000 mL) at 6° C. followed by addition of a solution of 50% ethyl acetate solution of 1-propanephosphonic acid cyclic anhydride (155.58 g, 145.54 mL, 244.48 mmol) over 15 minutes. Stir the mixture at 10° C. for 30 minutes and later at room temperature for 60 minutes. Cool to 15° C. and add water (200 mL), saturated aqueous sodium chloride (100 mL) and methanol (100 mL), stir the mixture for 5 minutes and separate the two phases. Extract the aqueous layer with a mixture of 10% methanol in ethyl acetate (a (2×1000 mL). Combine the organic layers, wash with saturated aqueous sodium chloride (2×250 mL), dry over sodium sulfate, filter and concentrate under reduced pressure but not to dryness. Cool at 4° C. and filter the solid, wash with cold ethyl acetate and methyl tert butyl ether. Heat the solid to reflux in ethanol (1300 mL) and cool the solution slowly at room temperature, keep the solution at room temperature for 12 hours and then 3 hours at 4° C. Filter the solid, wash with cold ethanol and methyl tert butyl ether to afford the title compound (61 g, 154.44 mmol). MS (m/z): 406 (M+1).

### Example 3

### N-{2-[(Pyridin-3-yloxy)acetyl]-1,2,3,4-tetrahydroisoquinolin-6-yl}pyridine-2-sulfonamide

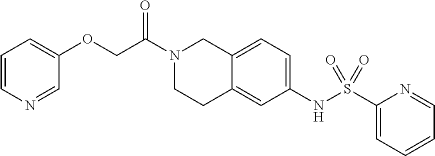 [SMILES: O=C(COc1cccnc1)N1CCc2cc(NS(=O)(=O)c3ccccn3)ccc2C1]

Add bis(2-oxo-3-oxazolidinyl)phosphonic chloride (406.7 mg, 1.6 mmol) and triethylamine (678.3 mg, 932.6 μL, 6.7 mmol) to a solution of N-(1,2,3,4-tetrahydroisoquinolin-6-yl)pyridine-2-sulfonamide hydrochloride (482.0 mg, 1.3 mmol) and 2-(3-pyridyloxy)acetic acid (203.9 mg, 1.3 mmol) in dimethylformamide (7 mL) and stir the mixture at room temperature for 3 hours. Add water and extract with dichloromethane twice, dry over anhydrous magnesium sulfate, filter and concentrate under reduced pressure. Pre-purify by silica gel chromatography, eluting with dichloromethane:methanol (95:5). Purify the crude material by Supercritical Fluid Chromatography (Luna Hilic column) eluting with a mobile phase of CO₂/methanol (15% to 30% methanol over 5.5 minutes at 100 g/minute) to afford the title compound (256 mg, 0.43 mmol). MS (m/z): 425 (M+1).

The following compounds are prepared essentially by the method of Example 3.

| Ex. | | | MS |
|---|---|---|---|
| No | Chemical Name | Structure | (m/z) |
| 4 | N-{2-[(Pyridin-3- yloxy)acetyl]-1,2,3,4- tetrahydroisoquinolin- 6-yl}tetrahydro-2H- pyran-4-sulfonamide | 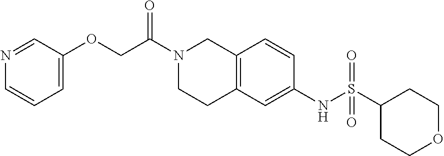 [SMILES: O=C(COc1cccnc1)N1CCc2cc(NS(=O)(=O)C3CCOCC3)ccc2C1] | 432 (M + 1) |
| 5 | 3,3,3-Trifluoro-N-{2- [(pyridin-3- yloxy)acetyl]-1,2,3,4- tetrahydroisoquinolin- 6-yl}propane-1- sulfonamide | 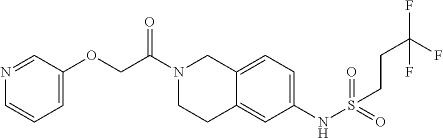 [SMILES: O=C(COc1cccnc1)N1CCc2cc(NS(=O)(=O)CCC(F)(F)F)ccc2C1] | 444 (M + 1) |

### Example 6

### 1-Cyclopropyl-N-{2-[(pyridin-3-yloxy)acetyl]-1,2,3,4-tetrahydroisoquinolin-6-yl}methanesulfonamide

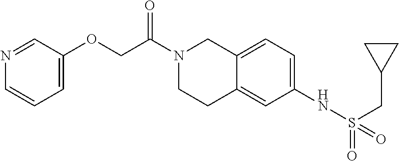 [SMILES: O=C(COc1cccnc1)N1CCc2cc(NS(=O)(=O)CC3CC3)ccc2C1]

Add O-(7-azabenzotriazol-1-yl)-N,N,N′,N′-tetramethyluronium hexafluorophosphate (584.56 mg, 1.81 mmol) and triethylamine (306.11 mg, 421.63 μL, 3.02 mmol) to a solution of 1-cyclopropyl-N-(1,2,3,4-tetrahydroisoquinolin-6-yl)methanesulfonamide (322.30 mg, 1.21 mmol) and 2-(3-pyridyloxy)acetic acid (185.30 mg, 1.21 mmol) in dimethylformamide (6 mL). Stir the mixture at room temperature for 18 hours. Concentrate under reduced pressure. Pre-purify by an ion exchange chromatography, eluting with 10% methanol/dichloromethane followed by 2 N NH₃ in methanol. Concentrate the latter basic fraction and further purify the crude material by HPLC (XTerra@ MS C18 21×100 m) eluting with a mobile phase of 20 mM ammonium carbonate at pH 9 in water/ACN (20% to 40% ACN over 8 minutes at 25 mL/minute) to afford the title compound (203 mg, 0.51 mmol). MS (m/z): 402 (M+1).

### Example 7

### 2-Isopropoxy-N-[2-[2-(3-pyridyloxy)acetyl]-3,4-dihydro-1H-isoquinolin-6-yl]ethanesulfonamide

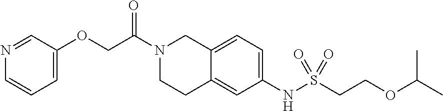 [SMILES: CC(C)OCCS(=O)(=O)Nc1ccc2c(c1)CCN(C(=O)COc1cccnc1)C2]

Add slowly 1-propanephosphonic acid cyclic anhydride (1.27 g, 1.19 mL, 1.99 mmol) to a solution of 2-isopropoxy-N-(1,2,3,4-tetrahydroisoquinolin-6-yl)ethanesulfonamide hydrochloride (444.00 mg, 1.33 mmol), 2-(3-pyridyloxy)acetic acid (223.35 mg, 1.46 mmol), triethylamine (402.51 mg, 554.42 μL, 3.98 mmol) and ethyl acetate (7 mL). Stir the mixture for 1 hour at room temperature. Add saturated aqueous potassium carbonate solution (50 mL) and extract twice with ethyl acetate, combine the organic layers, dry over anhydrous sodium sulfate, filter and concentrate under reduced pressure. Purify by prep-HPLC (Phenomenex Gemini® 10 Micron 50×150 mm C-18) (CH₃CN and water with 10 mM ammonium bicarbonate, 10% to 100% CH₃CN over 12 minutes at 120 mL/minutes) (2 injections). Combine the desired fractions and concentrate to afford the title compound (315 mg). MS (m/z): 434 (M+1).

### Example 8

### 1-(Oxetan-3-yl)-N-[2-[2-(3-pyridyloxy)acetyl]-3,4-dihydro-1H-isoquinolin-6-yl]piperidine-4-sulfonamide

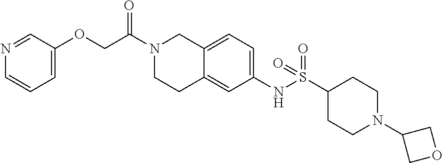 [SMILES: O=C(COc1cccnc1)N1CCc2cc(NS(=O)(=O)C3CCN(C4COC4)CC3)ccc2C1]

Add oxetan-3-one (275.68 mg, 3.83 mmol) to a solution of N-[2-[2-(3-pyridyloxy)acetyl]-3,4-dihydro-1H-isoquinolin-6-yl]piperidine-4-sulfonamide (1.22 g, 2.55 mmol) in dichloromethane (13 mL) and stir for one hour at room temperature then add sodium triacetoxy borohydride (807.19 mg, 3.83 mmol) and stir at room temperature overnight. Concentrate under reduced pressure. Pre-purify by ion exchange chromatography, eluting with methanol followed by 2 N NH₃ in methanol. Concentrate the latter basic fraction and further purify the crude material by HPLC (XBRIDGE™ C18 19×100 mm) eluting with a mobile phase of 20 mM ammonium carbonate at pH 9 in water/ACN (20% to 30% over 8 minutes at 25 mL/minute) to afford the title compound (553 mg, 1.14 mmol). MS (m/z): 487 (M+1).

### Example 9

### 1-Methyl-N-{2-[(pyridin-3-yloxy)acetyl]-1,2,3,4-tetrahydroisoquinolin-6-yl}piperidine-4-sulfonamide

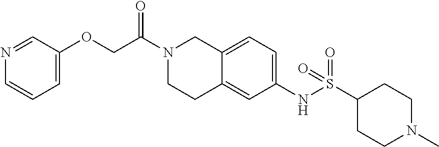 [SMILES: CN1CCC(S(=O)(=O)Nc2ccc3c(c2)CCN(C(=O)COc2cccnc2)C3)CC1]

Example 9 is prepared essentially by the method of Example 8. MS (m/z): 445 (M+1).

### Example 10

### N-[2-[2-(3-Pyridyloxy)acetyl]-3,4-dihydro-1H-isoquinolin-6-yl]-1-(tetrahydropyran-4-carbonyl)piperidine-4-sulfonamide

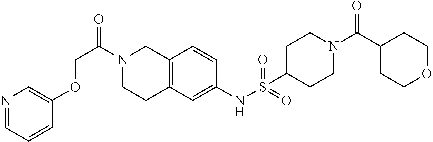 [SMILES: O=C(COc1cccnc1)N1CCc2cc(NS(=O)(=O)C3CCN(C(=O)C4CCOCC4)CC3)ccc2C1]

Add triethylamine (281.60 mg, 387.88 μL, 2.78 mmol) to a solution of N-[2-[2-(3-pyridyloxy)acetyl]-3,4-dihydro-1H-isoquinolin-6-yl]piperidine-4-sulfonamide (512.00 mg, 927.61 μmol) in dichloromethane (30 mL), stir for 30 minutes and add a solution of tetrahydro-pyran-4-carbonyl chloride (165.40 mg, 1159.65 μL, 11 mmol,) in dichloromethane (1 mL) dropwise at 0° C. and stir the mixture at that temperature for 30 minutes. Add dichloromethane (5 mL) and add saturated aqueous sodium carbonate solution (15 mL), separate the organic layers, dry over anhydrous sodium sulfate, filter and concentrate under reduced pressure. Purify by HPLC eluting with a mobile phase of 10 mM ammonium bicarbonate at pH 9 in water/ACN (10% to 100% CH₃CN over 10 minutes at 60 mL/minute) and evaporate the right fraction and triturate with ethyl ether, and filter to afford the title compound (190.40 mg, 0.5 mmol). MS (m/z): 543 (M+1).

### Example 11

### 3-Hydroxy-3-methyl-N-[2-[2-(3-pyridyloxy)acetyl]-3,4-dihydro-1H-isoquinolin-6-yl]pentanamide

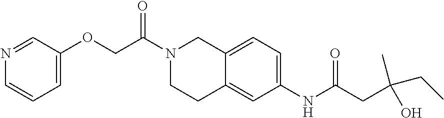 [SMILES: CCC(C)(O)CC(=O)Nc1ccc2c(c1)CCN(C(=O)COc1cccnc1)C2]

Add O-(7-azabenzotriazol-1-yl)-N,N,N′,N′-tetramethyluronium hexafluorophosphate (434.80 mg, 1.14 mmol) and triethylamine (154.28 mg, 212.51 μL, 1.52 mmol) to a solution of 3-hydroxy-3-methyl-N-(1,2,3,4-tetrahydroisoquinolin-6-yl)pentanamide (200.00 mg, 0.76 mmol) and 2-(3-pyridyloxy)acetic acid (116.74 mg, 0.76 mmol) in dimethylformamide (3.81 mL). Stir the mixture at room temperature for 18 hours. Concentrate under reduced pressure. Pre-purify by ion exchange chromatography, eluting with 10% methanol/dichloromethane followed by 2 N NH₃ in methanol. Concentrate the latter basic fraction and further purify the crude material by HPLC (XTerra MS C18 21×100 mm) eluting with a mobile phase of 20 mM ammonium carbonate at pH 9 in water/ACN (30% to 50% ACN over 8 minutes at 25 mL/minute) to afford the title compound (86 mg, 0.22 mmol). MS (m/z): 398 (M+1).

The following compounds are prepared essentially by the method of Example 11.

| Ex. | | | MS |
|---|---|---|---|
| No | Chemical name | Structure | (m/z) |
| 12 | 4,4,4-Trifluoro-3- hydroxy-3-methyl- N-[2-[2-(3- pyridyloxy)acetyl]- 3,4-dihydro-1H- isoquinlin-6- yl]butanamide | 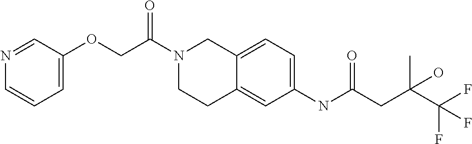 [SMILES: CC(O)(CC(=O)Nc1ccc2c(c1)CCN(C(=O)COc1cccnc1)C2)C(F)(F)F] | 438 (M + 1) |
| 13 | N-[2-[2-(3- Pyridyloxy)acetyl]- 3,4-dihydro-1H- isoquinolin-6-yl]-2- tetrahydropyran-2- yl-acetamide (isomer 2) | 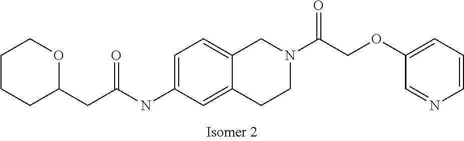 [SMILES: O=C(CC1CCCCO1)Nc1ccc2c(c1)CCN(C(=O)COc1cccnc1)C2] | 410 (M + 1) |
| 14 | 3,3-Dimethyl-N-{2- [(pyridin-3- yloxy)acetyl]- 1,2,3,4- tetrahydroiso- quinolin-6-yl}butanamide | 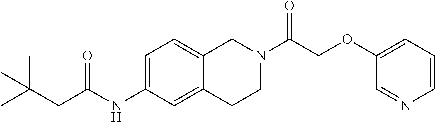 [SMILES: CC(C)(C)CC(=O)Nc1ccc2c(c1)CCN(C(=O)COc1cccnc1)C2] | 382 (M + 1) |
| 15 | 3-Hydroxy-3- methyl-N-{2- [(pyridin-3- yloxy)acetyl]- 1,2,3,4- tetrahydroiso- quinolin-6-yl} butanamide | 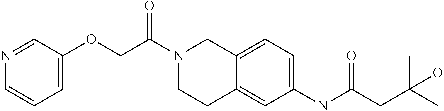 [SMILES: CC(C)(O)CC(=O)Nc1ccc2c(c1)CCN(C(=O)COc1cccnc1)C2] | 384 (M + 1) |

### Example 16

### 2-(Pyridin-3-yloxy)-1-[6-{[4-(tetrahydro-2H-pyran-4-ylcarbonyl)piperazin-1-yl]methyl}-3,4-dihydroisoquinolin-2(1H)-yl]ethanone

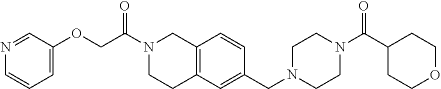 [SMILES: O=C(COc1cccnc1)N1CCc2cc(CN3CCN(C(=O)C4CCOCC4)CC3)ccc2C1]

Add triethylamine (144.36 mg, 198.85 μL, 1.43 mmol) to a solution of [4-(1,2,3,4-tetrahydroisoquinolin-6-ylmethyl)piperazin-1-yl]-tetrahydropyran-4-yl-methanone (245.00 mg, 0.71 mmol), O-(7-azabenzotriazol-1-yl)-N,N,N′,N′-tetramethyluronium hexafluorophosphate (406.84 mg, 1.07 mmol), 2-(3-pyridyloxy)acetic acid (142.01 mg, 0.92 mmol) in dimethylformamide (3.57 mL) and stir at room temperature overnight. Add dichloromethane and (30 mL) wash with saturated aqueous sodium chloride (30 mL), separate the organic layers, dry over anhydrous sodium sulfate, filter and concentrate under reduced pressure. Pre-purify by ion exchange chromatography, eluting with methanol followed by 2 N NH₃ in methanol. Purify by supercritical fluid chromatography (Luna Hilic column) eluting with a mobile phase of CO₂/methanol (15% to 30% methanol over 5.5 minutes at 100 g/minute) and evaporate the right fraction and triturate with ethyl ether, and filter to afford the title compound (23 mg, 0.05 mmol). MS (m/z): 479 (M+1).

The following compounds are prepared essentially by the method of Example 16.

| Ex. No | Chemical name | Structure | MS (m/z) |
|---|---|---|---|
| 17 | 2-(Morpholin-4-yl)-1- [4-({2-[(pyridin-3-yloxy) acetyl]-1,2,3,4-tetra- hydroisoquinolin-6-yl} methyl)piperazin- 1-yl]ethanone | 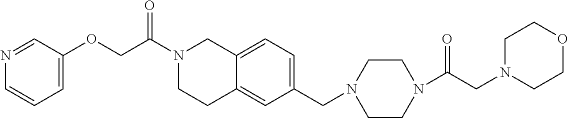 [SMILES: O=C(CN1CCOCC1)N1CCN(Cc2ccc3c(c2)CCN(C(=O)COc2cccnc2)C3)CC1] | 494 (M + 1) |
| 18 | 3-Hydroxy-3-methyl-1- [4-({2-[(pyridin-3-yloxy) acetyl]-1,2,3,4-tetrahydro- isoquinolin-6-yl}methyl) piperazin-1-yl]butan-1-one | 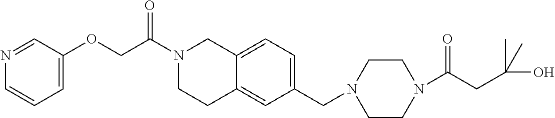 [SMILES: CC(C)(O)CC(=O)N1CCN(Cc2ccc3c(c2)CCN(C(=O)COc2cccnc2)C3)CC1] | 467 (M + 1) |
| 19 | 2-(Pyridin-3-yloxy)-1-[6-{[4- (tetrahydro-2H-pyran-4- ylacetyl)piperazin-1-yl]methyl}- 3,4-dihydroisoquinolin-2(1H)- yl]ethanone | 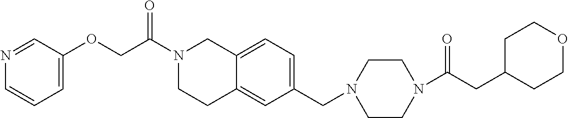 [SMILES: O=C(CC1CCOCC1)N1CCN(Cc2ccc3c(c2)CCN(C(=O)COc2cccnc2)C3)CC1] | 493 (M + 1) |
| 20 | 2-(3-Pyridyloxy)-1-[6-[1-[4- (tetrahydropyran-4-carbonyl) piperazin-1-yl]ethyl]-3,4- dihydro-1H-isoquinolin- 2-yl]ethanone (isomer 2) | 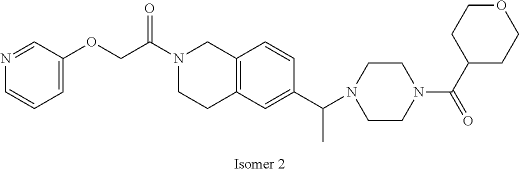 [SMILES: CC(c1ccc2c(c1)CCN(C(=O)COc1cccnc1)C2)N1CCN(C(=O)C2CCOCC2)CC1] | 493 (M + 1) |

### Example 21

### 2-Hydroxy-2-methyl-N-[2-[2-(3-pyridyloxy)acetyl]-3,4-dihydro-1H-isoquinolin-6-yl]propane-1-sulfonamide crystalline anhydrous free base

Add 2-hydroxy-2-methyl-N-[2-[2-(3-pyridyloxy)acetyl]-3,4-dihydro-1H-isoquinolin-6-yl]propane-1-sulfonamide (Example 1, 256 mg) in ethyl acetate (4 mL) and slurry at 1000 rpm at room temperature overnight to provide a white slurry. Filter the slurry by vacuum filtration, dry the solid in place on filter under vacuum and air stream for 10 minutes to provide the title compound (245 mg, 95.7% yield).

The X-ray diffraction (XRD) patterns of crystalline solids are obtained on a Bruker D4 Endeavor X-ray powder diffractometer, equipped with a CuKa source λ=1.54060 Å) and a Vantec detector, operating at 35 kV and 50 mA. The sample is scanned between 4 and 40° in 2θ, with a step size of 0.009° in 20 and a scan rate of 0.5 seconds/step, and with 0.6 mm divergence, 5.28 fixed anti-scatter, and 9.5 mm detector slits. The dry powder is packed on a quartz sample holder and a smooth surface is obtained using a glass slide. The crystal form diffraction patterns are collected at ambient temperature and relative humidity. It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. Furthermore, it is also well known in the crystallography art that for any given crystal form the angular peak positions may vary slightly. For example, peak positions can shift due to a variation in the temperature or humidity at which a sample is analyzed, sample displacement, or the presence or absence of an internal standard. In the present case, a peak position variability of 0.2 in 2θ will take into account these potential variations without hindering the unequivocal identification of the indicated crystal form. Confirmation of a crystal form may be made based on any unique combination of distinguishing peaks (in units of ° 2θ), typically the more prominent peaks. The crystal form diffraction patterns, collected at ambient temperature and relative humidity, are adjusted based on NBS standard reference material 675 (mica) with peaks at 8.853 and 26.774 degrees 2-theta.

Thus, a prepared sample of the crystalline free base is characterized by an X-ray diffraction pattern using CuKa radiation as having diffraction peaks (2-theta values) as described in Table 1 below, and in particular having peaks at 17.97 in combination with one or more of the peaks selected from the group consisting of 21.59, 18.53, and 14.96; with a tolerance for the diffraction angles of 0.2 degrees.

TABLE 1

| | Angle (°2-Theta) +/− | Relative Intensity (% of most intense |
|---|---|---|
| Peak | 0.2° | peak) |
| 1 | 8.96 | 16.40 |
| 2 | 11.00 | 22.70 |
| 3 | 14.96 | 35.00 |
| 4 | 16.26 | 20.50 |
| 5 | 16.81 | 29.00 |
| 6 | 17.97 | 100.00 |
| 7 | 18.53 | 50.00 |
| 8 | 21.59 | 87.10 |
| 9 | 22.90 | 23.10 |
| 10 | 23.82 | 23.80 |

Differential scanning calorimetry (DSC) analyses are carried out on a TA Instruments DSC unit Q2000. Samples are heated in crimped aluminum pans from 25 to 300° C. at 10° C./min with a nitrogen purge of 50 mL/min. The DSC temperature is calibrated with indium standard, onset of 156.3-156.9° C. This crystalline anhydrous free base displays a melting point onset at 164.06° C. by DSC.

### Example 22

### 2-Methoxy-N-[2-[2-(3-pyridyloxy)acetyl]-3,4-dihydro-1H-isoquinolin-6-yl]ethanesulfonamide crystalline anhydrous free base

Add triethylamine (306.11 mg, 0.42 ml, 3.02 mmoles) to a solution of 2-methoxy-N-(1,2,3,4-tetrahydroisoquinolin-6-yl) ethanesulfonamide (327.13 mg, 1.21 mmoles), 2-(3-pyridyloxy)acetic acid (185.30 mg, 1.21 mmoles), O-(7-azabenzotriazol-1-yl)-N,N,N′,N′-tetramethyluronium hexafluorophosphate (584.56 mg, 1.82 mmol) in 6 ml of dimethylformamide and stir overnight at room temperature. Concentrate under reduced pressure. Pre-purify by an ion exchange chromatography, eluting with methanol followed by 2 N NH₃ in methanol. Concentrate the latter basic fraction and further purify the crude material by HPLC (XTerra® MS C18 19×100 m), eluting with a mobile phase of 20 mM ammonium carbonate at pH 9 in water/ACN (15% to 35% ACN over 8 minutes at 25 mL/minute) to afford the title compound (130 mg, 0.32 mmol). MS (m/z): 406 (M+1).

The X-ray diffraction (XRD) patterns of crystalline solids are obtained as in essentially the same manner as described in Example 21 above.

Thus, a prepared sample of the crystalline free base is characterized by an X-ray diffraction pattern using CuKa radiation as having diffraction peaks (2-theta values) as described in Table 2 below, and in particular having peaks at 24.21 in combination with one or more of the peaks selected from the group consisting of 15.73, 18.95, and 18.28; with a tolerance for the diffraction angles of 0.2 degrees.

TABLE 2

| | Angle (°2-Theta) +/− | Relative Intensity (% of most intense |
|---|---|---|
| Peak | 0.2° | peak) |
| 1 | 10.42 | 8.60 |
| 2 | 15.73 | 59.30 |
| 3 | 16.51 | 33.70 |
| 4 | 17.24 | 25.30 |
| 5 | 18.28 | 33.90 |
| 6 | 18.95 | 48.20 |
| 7 | 23.43 | 14.80 |
| 8 | 24.21 | 100.00 |
| 9 | 26.08 | 23.20 |
| 10 | 27.32 | 15.20 |

### Biological Assays

It has been reported in the literature that NAMPT is over-expressed in several types of tumor cells including breast cancer, gastric cancer, colorectal cancer, liver cancer, renal cancer, brain cancer, melanoma, prostate cancer, NSCLC, and others; and its expression appears to be associated with tumor progression. See, for example, Bi, T. Q., et al., Oncol. Rep. 26, 1251-7, 2011; Hufton, S. E., et al., FEBS Lett. 463, 77-82, 1999; Van Beijnum. J. R., et al., Int. J. Cancer 101, 118-127, 2002; Wang, B., et al., Oncogene 30, 907-21, 2011; Nakajima, T. E., et al., J. Gastroenterol. 44, 685-90, 2009; Wang, B., et al., Oncogene 30, 907-21, 2011; Okumura S, et al., J Thorac Oncol. 7:49-56, 2012; Maldi, E. et al., Pigment Cell & Melanoma Research (published online October 2012); Bajrami I, et al., EMBO Mol Med. 4:1087-96, 2012; Zhang, L Q, et al., J Bioanal Biomed. 3: 013-025, 2011; Watson, M., et al., Mol. Cell. Biol. 29, 5872-88, 2009; Wieser V, et al., Digestive Diseases, 30(5):508-13. 2012; van Horssen R, et al., Cell Mol Life Sci. 70(12):2175-90, 2013; Drevs J, et al., Anticancer Res. 23:4853-4858, 2003; Zoppoli G, et al., Exp Hematol. 38(11):979-88, 2010; Aleskog A, et al., Anticancer Drugs, 12:821-827, 2001.

The following assays demonstrate that Examples 1 through 20, inhibitors of NAMPT, inhibit NAMPT catalytic activity. The results of the following assays also demonstrate that Examples 1 through 20 have in vitro cellular activities against the target, NAMPT, in cancer cells as the treatment of cancer cells with these compounds decreases their NAD⁺ formation and cell viability. Additionally, certain compounds of the present invention lead to the attenuation of glycolysis as indicated by the increase in the glycolytic intermediates before and at the glyceraldehyde3-phosphate dehydrogenase step and the decrease in the glycolytic intermediates after the glyceraldehyde3-phosphate dehydrogenase step. Attenuation of glycolysis leads to depletion of ATP and retardation of tumor cell growth. The results of the following assays also demonstrate that certain compounds of the present invention have in vivo activities against the target, NAMPT, in tumor xenograft as indicated by the decreased NAD⁺ formation. Furthermore, certain compounds of the present invention inhibit the growth of different tumor xenografts.

### NAMPT Biochemical Assay

The purpose of this assay is to measure the ability of a compound to inhibit NAMPT catalytic activity. Reaction mixtures (25 μL) containing 50 mM HEPES at pH 7.5, 50 mM NaCl, 1 mM DTT, 0.005% TRITON® X-100, 1.5 μM phosphoribosyl-pyrophosphate, 0.5 μM nicotinamide (NAM), 1.5 nM NAMPT, 2.5 mM ATP, 1.25 mM MgCl₂, 4% (v/v) DMSO and compounds after a ten-point series dilution from either 1 μM to 50 pM or 0.1 μM to 5 pM (final) are prepared. The reaction mixtures are incubated at room temperature for 2 hours. The reaction is terminated by the addition of ACN (25 μL) containing nicotinamide mononucleotide-d₄ (NMN-d4) as an internal standard (final concentration: 5 μM). The formation of nicotinamide mononucleotide (NMN) is quantified by a Liquid Chromatography-Mass Spectrometry (LC-MS) method as follows: NMN is analyzed on a Thermo Hypercarb Javelin column (2.1×20 mm, 5 μm) with an injection volume of 5 μL and a flow rate of 1 mL/minute using 0.1% formic acid for the mobile phase A and ACN for the mobile phase B. The gradient is as follows: 0 minutes, 0% B; 0.3 minutes, 0% B; 1.5 minutes, 35% B; 1.51 minutes, 95% B; 2.0 minutes, 95% B, 2.01 minutes, 0% B, 3 minutes, stop. A positive control group (enzyme and DMSO, but no compound) is used to measure minimum inhibition (0%) of NMN formation. Percent inhibition of compound treated groups is calculated relative to the minimum inhibition group. The relative IC₅₀ for each compound is calculated from a dose response study and is the concentration necessary to achieve 50% inhibition at this time point using the above disclosed ranges of 1 μM to 50 pM (final). The data generated from the dose-response studies is fit to a four-parameter logistic equation using ACTIVITYBASE 4.0 Equation 205. The results of this assay demonstrate that Examples 1 through 20 inhibit NAMPT catalytic activity, i.e. the compounds of these examples inhibit NAMPT with an IC₅₀ of equal or less than 16.7 nM. For example, Example 1 and Example 2 have an IC₅₀ value of 3.1 and 1.1 nM, respectively.

### Assay for NAD⁺/NMN Levels in A2780 Cells

The purpose of this assay is to demonstrate the ability of a compound to inhibit NAMPT activity required for the biosynthesis of NAD⁺/NMN in A2780 tumor cells. A2780 (the NCI-DCTD Tumor repository) tumor cells, an ovarian cancer cell line, are cultured in RPMI 1640 (SH30255.01, Hyclone) supplemented with 10% FBS. Cells are seeded into a 96-well culture plate (8×10⁴ cells/well) and incubated at 37° C. in 5% CO₂ for 4 hours, and then treated with a compound of the present invention (1 μM to 0.002 μM or 10 nM to 0.02 nM depending on the potency of each compound) for 24 hours. FK866 (100 nM) is also included as a positive control for maximum inhibition (100%). Each compound is tested 1-4 times in this assay.

To assess NAD⁺/NMN levels in the cell, A2780 cells grown in the above referenced 96-well plates are lysed with RIPA buffer (Pierce) followed by addition of 50 μL of 0.2 N HCl. The resulting cell lysates are incubated at 60° C. for 10 minutes and neutralized with 50 μL of 0.2 N NaOH. After centrifugation at 2000×g for 15 minutes, the supernatants (50 μL) are collected. The NAD⁺/NMN assays are described by Putt and Hergenrother (Putt, K. S, and Hergenrother, P. J., An enzymatic assay for poly(ADP-ribose) polymerase-1 (PARP-1) via the chemical quantitation of NAD⁺: application to the high-throughput screening of small molecules as potential inhibitors. Analytical Biochemistry, 2004, 326, 78-86) with modifications. The resulting lysates are mixed with 20 μL of 0.2 N KOH and 20 μL of 20% acetophenone, and incubated at 90° C. for 10 minutes followed by addition of 90 μL of formic acid. After incubation at 90° C. for 10 minutes, the resulting preparations are measured for their fluorescence at the excitation and emission wavelength of 360 and 450 nm, respectively as described by Putt and Hergenrother (2004). This assay demonstrates that Examples 1 through 20 inhibit NAMPT-mediated NAD⁺/NMN formation in A2780 tumor cells with IC₅₀ values of equal or less than 195 nM. For example, Example 1 and Example 2 have an averaged IC₅₀ value of 2.6±1.4 nM (SD, n=5) and 5.7 nM, ±3.3 (SD, n=4), respectively.

### Cell Proliferation Assay ±NA (Nicotinic Acid)

The purpose of this assay is to measure the ability of a compound to inhibit proliferation of endometrium, kidney, adrenal gland, and autonomic ganglia cancer cell lines dependent on the NAMPT-mediated NAD⁺ formation in the presence or absence of NA (10 μM) in vitro. One day before the planned initiation of compound treatment portion of the assay, one vial of assay ready frozen cells is thawed, and the cells are grown overnight in the media as shown in Table 3 under 5% CO₂ at 37° C. Then, the cell layer is briefly rinsed with 0.25% (w/v) Trypsin-0.038% (w/v) EDTA solution followed with the addition of 3.0 ml of Trypsin-EDTA solution. Once the cell layer is dispersed, 8.0 ml of complete growth medium (Table 3) is added and cells are aspirated by gently pipetting. The cell suspension is transferred to a centrifuge tube and centrifuged at 800-1000 rpm for 3-5 minutes. The supernatant is discarded using a vacuum pump. The cell pellet is suspended in a complete medium by gently pipetting. The cell numbers are counted and adjusted to the appropriate density (Table 3). For cell lines to be tested for 48, 96, and 120 hrs, 100 μL of cell suspension is added to each well in a 96-well plate (white-walled clear bottom). For cell lines to be tested for 144 hrs, 200 μL of cell suspension is added. The plates are incubated at 37° C. overnight. The next day in a separate plate, a ten-point compound dilution series (3-fold each) for a compound (from 2.0 μM to 0.0001 μM) is prepared in growth media containing 0.5% DMSO (v/v) without or with 10 μM NA (final). Then, 0.5 μL or 1 μL of compound after a ten-point series dilution is added to each well containing 100 or 200 μL of cell suspension. The cell plates are covered and incubated for 48, 96, 120, or 144 hours under at 37° C. After the incubation, the cell plates are equilibrated to room temperature for approximately 30 minutes. Before the assay, the CellTiter-Glo Buffer (Promega) is thawed and equilibrated to room temperature. The lyophilized CellTiter-Glo substrate (Promega) is also equilibrated to room temperature. The appropriate volume of CellTiter-Glo Buffer (Promega) is transferred into an amber bottle containing CellTiter-Glo substrate to reconstitute the lyophilized enzyme/substrate mixture, which forms the CellTiter-Glo Reagent. The CellTiter-Glo Reagent (100 μl) is added to the cell plates. The plates are shaken on an orbital shaker for 2 minutes to induce cell lysis and then incubated at room temperature for 10 minutes. The bottom of each plate is pasted with a white back seal and luminescence is recorded using a Flexstation 3 with the following settings: Luminescence and integration time of 500 ms.

This assay demonstrates that Example 1 inhibits proliferation of a number of kidney, endometrium, adrenal gland, and autonomic ganglia cancer cell lines in the presence or absence of NA in vitro. This assay also demonstrates that the anti-proliferative activity of Example 1 against some of the cancer cell lines tested is rescued or reversed by the addition of 10 μM NA to the growth medium as indicated by the increased IC₅₀ values to >2.0 μM, showing that Example 1 specifically inhibits NAMPT in the cell, while the anti-proliferative activity of Example 1 against a number of other cancer cell lines tested is not rescued or reversed by the addition of 10 μM NA to the growth medium as indicated by the relatively unchanged IC₅₀ values. Therefore, this assay additionally demonstrates that a significant portion of the cancer types that the cancer cell lines represent does not express or expresses a very low level of NAPRT.

TABLE 3
Growth conditions and sources of cell lines

| | Cell | Treatment | | |
|---|---|---|---|---|
| Cell line | density | time (hr) | Growth medium | Sources |
| SW13 | 3000 | 72 | Leibovitz's L-15, 10% FBS | ATCC |
| NCI-H295 | 3500 | 144 | Dulbecco's medium: Ham's | ATCC |
| | | | F12, 50:50 mix | |
| | | | Insulin 0.005 mg/ml, | |
| | | | Transferrin 0.01 mg/ml, | |
| | | | Sodium selenite 30 nM, | |
| | | | Hydrocortisone 10 nM | |
| | | | beta-estradiol 10 nM, | |
| | | | HEPES 10 mM | |
| | | | L-glutamine 2 mM, fetal | |
| | | | bovine serum 2% | |
| CHP-212 | 3500 | 72 | DMEM:F12(1:1)+ 10% FBS | ATCC |
| TGW | 20,000 | 48 | MEM + 10% FBS | JCRB* |
| GOTO | 4000 | 96 | RPMI1640 mixed with | JCRB* |
| | | | EMEM (1; 1), 10% FBS | |
| HEC-1-A | 3500 | 72 | McCoy′5A + 10% FBS | SIBS* |
| SNG-M | 9000 | 72 | Ham's F-12 medium, 10% | ATCC |
| | | | FBS | |
| AN3-CA | 3000 | 72 | Eagle's Minimum Essential | ATCC |
| | | | Medium, 10% FBS | |
| KLE | 3000 | 72 | DMEM:F12 Medium, 10% | ATCC |
| | | | FBS | |
| COLO- | 3000 | 72 | RPMI 1640 + 2 mM | SIBS* |
| 684 | | | Glutamine + 10% FBS | |
| RL95-2 | 3000 | 72 | DMEM:F12 Medium, | ATCC |
| | | | 0.005 mg/ml insulin, 10% | |
| | | | FBS | |
| A704 | 3500 | 72 | EMEM + 10% FBS | ATCC |
| OS-RC-2 | 3500 | 72 | RPMI1640 + 10% FBS | SIBS* |
| SN12C | 5000 | 72 | RPMI 1640 10% FBS | SIBS* |
| 786-0 | 2000 | 48 | EMEM, 10% FBS | ATCC |
| Caki-1 | 2500 | 72 | McCoy′5A + 10% FBS | ATCC |
| TK10 | 5000 | 72 | RPMI 1640, 10% FBS | SIBS* |
| ACHN | 3500 | 72 | EMEM + 10% FBS | ATCC |
| A498 | 3000 | 48 | EMEM + 10% FBS | ATCC |
| U031 | 5000 | 72 | RPMI 1640 + 10% FBS | ATCC |

*JCRB: Japanese Collection of Research Bioresources; SIBS: Shanghai Institutes for Biological Sciences

TABLE 4
Anti-proliferative activity of Example 1 in different cancer cell lines

| | | IC50 (μM) | IC50 (μM) |
|---|---|---|---|
| CELL LINE | CANCER TYPE | NA (0.0 μM) | NA (10 μM) |
| SW13 | adrenal gland | 0.05 | 0.02 |
| NCI-H295 | adrenal gland | 0.10 | 0.06 |
| CHP-212 | autonomic ganglia | 0.07 | 0.07 |
| TGW | autonomic ganglia | 0.01 | 0.01 |
| GOTO | autonomic ganglia | 0.02 | 0.02 |
| HEC-1-A* | endometrium | >2 | >2 |
| SNG-M | endometrium | 0.02 | 0.02 |
| AN3-CA | endometrium | 0.02 | 0.02 |
| KLE | endometrium | 0.04 | >2 |
| COLO-684 | endometrium | 0.03 | >2 |
| RL95-2 | endometrium | 0.02 | 0.02 |
| A704 | kidney | 0.37 | >2 |
| OS-RC-2 | kidney | >2 | >2 |
| SN12C | kidney | 0.06 | 0.05 |
| 786-0 | kidney | 0.07 | 0.04 |
| Caki-1 | kidney | 0.13 | 0.11 |
| TK10 | kidney | 0.22 | 0.21 |
| ACHN | kidney | 0.11 | >2 |
| A498 | kidney | 1.84 | >2 |
| U031 | kidney | 0.06 | 0.06 |

*HEC-1A is tested in a growth medium containing nicotinic acid.

### A2780 Proliferation Assay ±NAM (Nicotinamide)

The purpose of this assay is to measure the ability of a compound to inhibit proliferation of A2780 cells (the NCI-DCTD Tumor repository) dependent on the NAMPT-mediated NAD⁺ formation in the presence or absence of higher concentrations of NAM (10 mM) in vitro. The A2780 cell proliferation assay uses assay-ready frozen cells. To prepare assay-ready frozen A2780 cells, A2780 cells, an ovarian cancer cell line, are cultured in growth media containing RPMI 1640 (Gibco 30-2001) supplemented with 10% FBS, in T-150 flasks for 3-4 days. Cells are then treated with 4 mL of 0.25% (v/v) trypsin for 1 minute (Hyclone SH30042). Trypsin-treated cells are then diluted with 10 mL of growth media, and the cell slurry is gently mixed and then decanted to a centrifuge tube. Cells are counted and then pelleted by centrifugation at 1400 rpm for 5 minutes. After centrifugation the supernatant is removed and the cell pellets are re-suspended in GIBCO® RECOVERY™ Cell Culture Freezing Medium (Invitrogen 12648-010) at 2-5×10⁶ cells/mL and then aliquoted at 1 mL volumes into cryovials. Cryovials are stored initially at −80° C. for 16 hours, and then transferred to liquid nitrogen for long term storage.

One day before the planned initiation of compound treatment portion of the assay, one vial of assay ready frozen cells is thawed, and the cells are washed with 50 mL of growth medium. Cells are counted and then diluted to 2.8×10⁴ cells/mL and then plated at a rate of 2500 cells/well (90 μL per well) to BD Poly-D-Lysine, 96 well black plates (BD Biocoat 35-4640). Plates are then covered and incubated overnight under 5% CO₂ at 37° C. The next day in a separate plate (V bottom Nunc 249946), a ten-point compound dilution series for each compound is prepared in growth media containing 2% DMSO (v/v, 0.2% final) with or without 100 mM NAM (10 mM final). Then 10 μL of compounds after a ten-point series dilution from either 2 or 0.1 μM (depending potency of each compound) to 50 pM or 5 pM (final) are added to the wells of the cell plates. Cell plates are covered and incubated for 72 hours under 5% CO₂ at 37° C.

On the day of the viability assessment, one vial of GF-AFC substrate (CELL TITER-FLUOR™ Cell Viability Assay Kit, Promega G6081) is vortexed and the substrate is transferred to one vial of thawed CELL TITER-FLUOR™ Assay Buffer. The resulting CELL TITER-FLUOR™ reagent is then vortexed well to thoroughly dissolve the substrate. The CELL TITER-FLUOR™ reagent is then diluted (1:2) in growth medium and 50 μL of diluted CELL TITER-FLUOR™ reagent is added to each well of the cell plate. The cell plate is covered and incubated under 5% CO₂ at 37° C. for 1-3 hours. Finally the cell plates are removed from the incubator and the well fluorescence is measured on an Envision® Multilabel Reader (Perkin Elmer, λ_{eX} 355/λₑₘ 495). Fluorescence from compound treated wells is compared to the no cell and no compound treatment control wells to calculate percent inhibition. The percent inhibition and ten-point compound concentration data is fit to a four-parameter logistic equation using ACTIVITYBASE 4.0 Equation 205. Each compound is tested 2-4 times in this assay. This assay demonstrates that Examples 1 through 20 inhibit proliferation of A2780 cells in the absence of NAM in vitro with IC₅₀ values below 677 nM. For example, Example 1 and Example 2 have an IC₅₀ of 11.8±3.0 nM (SD, n=4) and 34.3±14.4 nM (SD, n=3), respectively. The anti-proliferative activity of Example 1 and Example 2 against A2780 cancer cells is rescued or reversed by the addition of 10 mM nicotinamide to the growth medium as indicated by the increased IC₅₀ value to >0.1 μM, showing that Example 1 and Example 2 specifically inhibits NAMPT in the cell.

### Cell Viability Assay

The purpose of this assay is to measure the ability of a compound to reduce viability of different cancer cells dependent on the NAMPT-mediated NAD⁺ formation in vitro. HCC1937 (breast cancer) cells are cultured in RPMI-1640 supplemented with 10% FBS. Calu-6 (lung cancer) cells and MCF-7 (breast cancer) cells are cultured in Minimum Essential Medium (MEM) (Gibco11095) supplemented with 1 mM sodium pyruvate (Gibco 11360), 1% Non-Essential Amino Acids Solution (100×; Gibco 11140) and 10% FBS. NCI-H1155 (lung cancer) cells are cultured in Dulbecco's Modified Eagle Medium (DMEM) (Gibco 11965) with 10% FBS. Cells (2000/well for adherent cell and 10000/well for suspension cell) are seeded in 96-well plates, cultured overnight (≈18 hours), and treated in 2-3 replicates with a compound of the present invention (formulated in DMSO at concentrations from 1.000 μM to 0.051 nM) for 72 hours. The cells are also treated with staurosporine (10 μM) as a positive control and 0.1% DMSO as a negative control. Cell viability is analyzed by using an assay kit (CYTOTOX-GLO™ Cytotoxicity Assay kit, Promega) according to the manufacturer's instructions as follows. 50 μL of CYTOTOX-GLO™ Cytotoxicity Assay reagent is added to each well. The plate is mixed briefly by orbital shaking. The plate is incubated for 15 minutes at room temperature. Luminescence is measured using a Wallac Victor3 V 1420 Multilabel Counter (Perkin Elmer), referred to as dead cell luminescence. Add 50 μL of Lysis Reagent to each well, and the plate is mixed briefly by orbital shaking. After the plate is incubated at room temperature for 15 minutes, luminescence is measured using the plate reader, referred to as total luminescence. Viable cell luminescence (CPS) is calculated by subtracting the dead cell luminescence from the total luminescence Inhibition of cell viability is calculated based on the equation as follows:

Inhibition (%)=(CPS_{negative}−CPSₛₐₘₚₗₑ)/(CPS_{negative}−CPSₚₒₛᵢₜᵢᵥₑ)*100 where CPS is the luminescence of viable cells.

TABLE 5
IC₅₀ of Example 1 and Example 2 in cancer cells

| | NCI-H1155 | Calu-6 | HCC1937 | MCF-7 |
|---|---|---|---|---|
| | IC₅₀ (μM) | IC₅₀ (μM) | IC₅₀ (μM) | IC₅₀ (μM) |
| Example 1 | 0.018 ± 0.003 (SD) | 0.049 ± 0.008 (SE) | 0.333 ± 0.032 (SE) | 0.389 ± 0.283 (SE) |
| Example 2 | 0.037 ± 0.016 (SD) | 0.121 ± 0.029 (SD) | 1.04 ± 0.664 (SD) | 0.352 ± 0.252 (SD) |

*Example 1 is tested 5 times (2-3 replicates each) in NCI-H1155, and once (3 replicates) in Calu 6, HCC1937, and MCF-7; Example 2 is tested 5 times (2-3 replicates each) in NCI-H1155, twice (2-3 replicates) in Calu6 and HCC1937; and 3 times (2-3 replicates each) in MCF-7.

This assay demonstrates that Example 1 and Example 2 induce cell death in NCI-H1155, Calu-6, HCC1937, and MCF-7 cell lines.

### LC-MS Analysis of NAD⁺ and Carbohydrate Metabolites in A2780 Cancer Cells

The purpose of this assay is to measure effects of NAMPT inhibitors on NAD⁺ formation in cancer cells. LC-MS analysis of NAD⁺ metabolites: nicotinamide mononucleotide (NMN), NAD⁺, reduced nicotinamide adenine dinucleotide (NADH), and nicotinamide adenine dinucleotide phosphate (NADP) is performed on an HPLC system coupled to a Thermo Quantum Ultra triple quadrupole mass spectrometer operated in positive heated electrospray mode with selected reaction monitoring detection. For cell extracts, 50 μL of extract and 10 μL of 10 μM internal standard (IS) solution is transferred to a 96-well plate, dried under nitrogen and reconstituted in 50 μL of water. For tissue extracts, 20 μL of extract and 10 μL of IS solution are dried and reconstituted in 50 μL water. The IS solution contains 10 μM nicotinamide-d₄ (C/D/N Isotopes), nicotinic acid-d₄ (C/D/N Isotopes), nicotinamide mononucleotide-d₄ (prepared by custom synthesis) and nicotinamide 1,N⁶-ethenoadenine dinucleotide in methanol. The metabolites are separated on a Waters XBRIDGE™ Amide (2.1×50 mm, 3 μm) with an injection volume of 10 μL and a flow rate of 1 mL/minute using 10 mM ammonium acetate in 95% acetonitrile for mobile phase A and 10 mM ammonium acetate in 50% acetonitrile for mobile phase B. The gradient is as follows: 0 minutes, 0% B; 2.5 minutes, 70% B; 2.51 minutes, 100% B; 2.8 minutes, 100% B; 2.81 minutes, 0% B, 3.6 minutes, 0% B.

The purpose of this assay is to measure effects of NAMPT inhibitors on the levels of metabolites such as glucose-6-phosphate/fructose-6-phosphate/fructose-1-phosphate collectively as hexose phosphate (HP), fructose-1,6-bisphosphate (FBP), glyceraldehyde-3-phosphate (G3P), dihydroxyacetone phosphate (DHAP), 3-phosphoglycerate (3PG), 2-phosphoglycerate (2PG), phosphoenoylpyruvate (PEP), gluconate-6-phosphate (GN6P), xylulose-5-phosphate (X5P), ribulose-5-phosphate (Ru5P), ribose-5-phosphate (R5P), sedoheptulose-7-phosphate/sedoheptulose-1-phosphate collectively as sedoheptulose phosphate (SP), erythrose-4-phosphate (E4P), and α-ketoglutarate (α-KG) derived from glycolysis, the TCA cycle, and the pentose-phosphate pathway in tumor cells. Cells (50,000/well) are grown as described above in 100 μL of Dulbecco's Modified Eagle Medium supplemented with 10% FBS (dialyzed) and 25 mM glucose, and treated with a compound in triplicates in the presence or absence of 10 μM nicotinic acid. After 24 hours of treatment, the growth medium is removed and 200 μL of 80% methanol is added to each well. After incubation at room temperature for 15 minutes, the resulting extracts are transferred to 96-deep-well plates and washed twice with 200 μL of 80% methanol/water. Then, the plates are heat sealed and stored at −80° C., or dried and reconstituted in 100 μL of 25 μM ethylenediaminetetraacetic acid and injected into LC-MS for analysis.

The LC-MS analysis for carbohydrate metabolites is performed as follows. Chromatographic separations are performed with an HPLC system, which is coupled to an AB Sciex triple quadrupole LC-MS mass spectrometer. Analytes with phosphates are analyzed as follows. The samples are dried and reconstituted in ACN/water solutions and are separated on a Phenomenex Luna amino HPLC column (2.1×30 mm 3 μm) under the conditions as described by Yuan et al. (Nature Protocols, 2012, 17, 872-881.). The mass spectrometer is operated under negative ESI MRM mode.

This assay demonstrates that certain compounds of the present invention inhibit NAD formation. For example, Example 1 demonstrates a dose-dependent inhibition of NAD formation. The NAD depletion demonstrated by certain compounds, for example, Example 1, leads to the attenuation of glycolysis at the G3P dehydrogenase step as indicated by the dose-dependent increase in the glycolytic intermediates (HP, FBP, and DHAP/G3P) before and at the G3P dehydrogenase step and the dose-dependent decrease in the intermediates (PEP, and PG) after the G3P dehydrogenase step. The attenuation of glycolysis demonstrated by certain compounds, for example, Example 1, subsequently results in the perturbation of other metabolic pathways such as the pentose phosphate pathway as the key intermediates including, for example, SP, are increased.

TABLE 6
Example 1 Inhibits NAD⁺ Formation, Attenuates Glycolysis, and
Alters the Pentose Phosphate Pathway in A2780 Cancer Cells (3 Replicates)

| | Metabolite Levels (μM) | | | | | Standard Error of Means (μM) | | | | |
|---|---|---|---|---|---|---|---|---|---|---|
| Example 1 (μM) | DHAP | FBP | FP | SP | NAD | DHAP | FBP | HP | SP | NAD |
| 10 | 5.5 | 39.7 | 17.1 | 2.8 | 0.008 | 0.39 | 4.46 | 1.11 | 0.14 | 0.001 |
| 3.333 | 5.2 | 43.0 | 18.8 | 3.4 | 0.007 | 0.10 | 2.43 | 1.18 | 0.14 | 0.001 |
| 1.111 | 5.4 | 38.6 | 18.0 | 3.3 | 0.008 | 0.26 | 2.10 | 0.55 | 0.08 | 0.001 |
| 0.37 | 5.0 | 40.9 | 17.2 | 3.2 | 0.010 | 0.66 | 2.40 | 0.89 | 0.16 | 0.000 |
| 0.123 | 3.8 | 34.3 | 15.1 | 2.6 | 0.008 | 0.23 | 1.95 | 0.96 | 0.13 | 0.002 |
| 0.0412 | 2.0 | 19.3 | 7.6 | 1.3 | 0.014 | 0.16 | 1.14 | 0.39 | 0.02 | 0.002 |
| 0.0137 | 1.0 | 7.2 | 3.0 | 0.5 | 0.027 | 0.08 | 0.75 | 0.31 | 0.06 | 0.002 |
| 0.00457 | 0.6 | 4.1 | 2.4 | 0.4 | 0.089 | 0.02 | 0.52 | 0.08 | 0.01 | 0.004 |
| 0.00152 | 0.6 | 3.0 | 2.1 | 0.3 | 0.244 | 0.04 | 0.20 | 0.15 | 0.01 | 0.008 |
| 0.000508 | 0.6 | 2.5 | 1.9 | 0.3 | 0.331 | 0.02 | 0.38 | 0.14 | 0.03 | 0.016 |
| 0 | 0.5 | 2.5 | 2.2 | 0.3 | 0.368 | 0.03 | 0.35 | 0.17 | 0.01 | 0.013 |

TABLE 7
Example 2 Inhibits NAD⁺ Formation, Attenuates Glycolysis, and
Alters the Pentose Phosphate Pathway in A2780 Cancer Cells (3 Replicates)

| | Metabolite Levels (μM) | | | | | Standard Error of Means (μM) | | | | |
|---|---|---|---|---|---|---|---|---|---|---|
| Example 2 (μM) | DHAP | FBP | HP | SP | NAD | DHAP | FBP | HP | SP | NAD |
| 10 | 7.7 | 21.5 | 10.9 | 2.1 | 0.02 | 0.44 | 0.55 | 0.58 | 0.07 | 0.001 |
| 3.333 | 7.6 | 23.4 | 12.8 | 2.5 | 0.02 | 0.10 | 0.78 | 0.08 | 0.06 | 0.001 |
| 1.111 | 7.3 | 24.1 | 11.8 | 2.5 | 0.017 | 0.31 | 0.62 | 0.57 | 0.11 | 0.001 |
| 0.37 | 7.1 | 22.8 | 11.0 | 2.2 | 0.022 | 0.40 | 0.71 | 0.04 | 0.03 | 0.002 |
| 0.123 | 7.1 | 15.5 | 7.2 | 1.3 | 0.023 | 0.14 | 0.66 | 0.15 | 0.05 | 0 |
| 0.0412 | 6.5 | 6.4 | 2.8 | 0.5 | 0.039 | 0.15 | 0.31 | 0.10 | 0.04 | 0.003 |
| 0.0137 | 6.3 | 3.2 | 1.9 | 0.3 | 0.087 | 0.30 | 0.39 | 0.15 | 0.01 | 0.003 |
| 0.00457 | 3.2 | 2.2 | 1.6 | 0.2 | 0.255 | 0.10 | 0.11 | 0.12 | 0.01 | 0.004 |
| 0.00152 | 0.9 | 1.8 | 1.6 | 0.2 | 0.457 | 0.08 | 0.25 | 0.12 | 0.01 | 0.003 |
| 0.000508 | 0.5 | 1.4 | 1.6 | 0.2 | 0.527 | 0.02 | 0.19 | 0.13 | 0.01 | 0.02 |
| 0 | 0.4 | 1.1 | 1.7 | 0.2 | 0.621 | 0.01 | 0.13 | 0.09 | 0.004 | 0.014 |

### IVTI Assay

The purpose of this assay is to measure the ability of a test compound to inhibit the NAMPT-mediated NAD⁺ formation in tumors in an animal model. A2780 cells (ATCC) are grown as described above for the NAD⁺ assay (Assay for NAD⁺/NMN Levels in A2780 Cells). The cells (5×10⁶/animal) are mixed with MATRIGEL® (1:1) and implanted subcutaneously into the rear flank of the mice (female nude mice, Harlan). The implanted tumor cells grow as solid tumors. The tumor volume and body weight are measured twice a week with a caliper. After tumor volume reaches approximately 300-500 mm³, animals are randomized and grouped into positive control (described herein; 5 animals/group) and compound treatment groups (5 animals/group). The compound (formulated in 20% of CAPTISOL® and 25 mM of phosphate buffer, pH 2) and positive control (20% of CAPTISOL® and 25 mM of phosphate buffer, pH 2) are administered by oral gavage. Compound doses are in the range of 0.10 to 25 mg/kg. Mice are sacrificed 17 hours after a single dose or 7 hours after second dose (19 hours after the first dose). Tumor tissues are harvested and homogenized as described below. Tumor tissues (˜100 mg each) are placed into in a tube (Lysing Matrix D tube, MPBio #6913-100) on dry ice and homogenized in an extraction buffer (0.8 mL each) (Biovision, cat#K337-100-1) for 45 seconds (3×15 seconds) using a Bio101 FastPrep FP120 homogenizer (setting 5). The resulting preparations (0.5 mL each) are filtered (with a 10K cutoff filter) to remove hemoglobin because red color interferes with the absorbance. Centrifuge the resulting preparations in accordance with the manufacturer's instructions (9500 RPM×40 minutes, Millipore). The flow through are collected and stored at −80° C. until they are assayed. In a 96-well plate, the collected samples (≈31 μL each) are diluted (1:8) into an extraction buffer (≈249 μL each) (BioVision, cat#337-100-1) to a final volume of about 280 μL. The resulting preparations (˜140 μL each) are transferred to another 96-well plate, which is heated to 60° C. for 30 minutes. The plate is cooled to room temperature for approximately 4 to 10 minutes and then centrifuged briefly. NAD⁺ quantitation is carried out using a NAD⁺/NADH cycling assay kit (BioVision, cat#337-100-1). The positive control (vehicle group) is used to measure the minimum inhibition (0%) of NAD⁺ formation. The percent inhibition of compound treated groups is calculated relative to the minimum inhibition groups. TED₅₀ is calculated from a dose response study and is the dose necessary to achieve 50% inhibition at this time point. This assay demonstrates the ability of a test compound to inhibit the NAMPT-mediated NAD⁺ formation in tumors in an animal model. For example, Example 1 has a TED₅₀ value of 2.56±0.37 mg/kg (SE) after 2 doses.

### LC-MS Analysis of NAD⁺ in A2780 and NCI-H1155 Tumor Xenografts

The purpose of this assay is to measure the effects of NAMPT inhibitors on NAD⁺ levels in vivo as described above. Tumors are grown and treated as described below for efficacy in xenograft tumor models below (7 animals/group). Each compound is also formulated as described for efficacy in xenograft tumor models below. Vehicle is 20% of CAPTISOL® and 25 mM of phosphate buffer, pH 2 without compound. After the completion of the treatment, tumor tissues (˜50 mg each) are homogenized in an ice cold extraction buffer (1 mL of 70% methanol/water; HPLC grade for 2 minutes in a tissue lyser II (QIAGEN™) at frequency 30 Hz. The resulting preparations are centrifuged at 14000×g for 6 minutes. The supernatant fractions (500 μL each) are collected and extracted with chloroform (0.5 mL). The aqueous fractions (0.3 mL each) are collected into a 96-well plate ready for LC-MS analysis. The LC-MS analysis of NAD metabolites is performed as described above.

This assay demonstrates that certain compounds of the present invention inhibit NAD⁺ formation in tumor xenografts. This assay also demonstrates Example 1 and Example 2 inhibit the target NAMPT not only in vitro in cancer cells and also in vivo in tumors as it reduces NAD⁺ levels in tumors.

TABLE 8
Example 1 and Example 2 Inhibit NAD⁺ Formation
in A2780 Tumor Xenografts

| | NAD levels | Standard Error of Means |
|---|---|---|
| Treatment group | (pmol/mg tissue) | (pmol/mg) |
| Vehicle | 56.30 | 9.48 |
| Example 1 5 mg/kg (BID) | 5.04 | 1.21 |
| Example 1 10 mg/kg (BID) | 2.55 | 0.63 |
| Example 1 20 mg/kg (BID) | 3.29 | 1.85 |
| Vehicle | 55.03 | 18.11 |
| Example 2 8 mg/kg (BID) | 3.97 | 1.09 |
| Example 2 16 mg/kg (BID) | 6.52 | 1.56 |
| Example 2 32 mg/kg (BID) | 6.38 | 0.91 |

TABLE 9
Example 1 and Example 2 Inhibit NAD⁺ Formation
in NCI-H1155 Tumor Xenografts

| | NAD levels | Standard Error of Means |
|---|---|---|
| Treatment group | (pmol/mg tissue) | (pmol/mg) |
| Vehicle | 20.08 | 2.39 |
| Example 1 2.5 mg/kg (BID) | 11.38 | 2.59 |
| Example 1 5 mg/kg (BID) | 7.48 | 1.50 |
| Example 1 10 mg/kg (BID) | 3.63 | 1.31 |
| Example 1 20 mg/kg (BID) | 1.82 | 0.35 |
| Vehicle | 57.97 | 10.65 |
| Example 2 8 mg/kg (BID) | 5.19 | 1.26 |
| Example 2 16 mg/kg (BID) | 1.98 | 0.63 |
| Example 2 32 mg/kg (BID) | 0.84 | 0.39 |

### Efficacy in Xenograft Tumor Models

The purpose of this assay is to measure reduction in tumor volume in response to test compound administration. A2780 and NCI-H1155 (NSCLC) cells are grown as described above for IVTI studies. Cells are harvested and injected subcutaneously onto the rear flank of nude mice. When tumors are established (7-21 days after implant), animals are randomized and grouped into control and test groups (7 animals/group). The test compound is formulated in 20% of CAPTISOL® and 25 mM of phosphate buffer, pH 2. Test compound and vehicle (20% of CAPTISOL® and 25 mM of phosphate buffer, pH 2 without compound) are administered by oral gavage. Tumor response is determined by tumor volume measurement (caliper) performed twice a week during the course of treatment and reported as percent of tumor volume of each treatment group divided by tumor volume of the vehicle control group. Example 1 and Example 2 demonstrate dose dependent anti-tumor activity in A2780 and NCI-H1155 xenograft tumor models. For example, Example 1 in H1155 tumor model, when dosed at 10 mg/kg (twice a day (BID) on a 4-day-on and 3-day-off schedule for 17 days, a T/C of 5.5 (P value<0.001 based on T-test) is achieved; when dosed at 20 mg/kg on the same schedule, a T/C of −81.1 (P value<0.001 based on T-test) is achieved. Example 2 in H1155 tumor model, when dosed at 8 mg/kg (twice a day (BID) on a 4-day-on and 3-day-off schedule for 17 days, a T/C of 5.2 (P value<0.001 based on T-test) is achieved; when dosed at 16 mg/kg on the same schedule, a T/C of −82.7 (P value<0.001 based on T-test) is achieved. Example 1 in A2780 tumor model, when dosed at 10 mg/kg (twice a day (BID) on a 4-day-on and 3-day-off schedule for 17 days, a T/C of 41.7 (P value<0.007 based on T-test) is achieved; when dosed at 20 mg/kg on the same schedule, a T/C of 2.4 (P value<0.001 based on T-test) is achieved. Example 2 in A2780 tumor model, when dosed at 8 mg/kg (twice a day (BID) on a 4-day-on and 3-day-off schedule for 17 days, a T/C of 40.5 (P value<0.063 based on T-test) is achieved; when dosed at 16 mg/kg on the same schedule, a T/C of 1.5 (P value<0.001 based on T-test) is achieved. This data demonstrates that Example 1 and Example 2 inhibit tumor xenograft growth in this tumor model.

The compounds of the present invention are preferably formulated as pharmaceutical compositions administered by a variety of routes. More preferably, such compositions are for oral or intravenous administration. Such pharmaceutical compositions and processes for preparing same are well known in the art. See, e.g., REMINGTON: THE SCIENCE AND PRACTICE OF PHARMACY (D. Troy, et al., eds., 21^{st} ed., Lippincott Williams & Wilkins, 2005). Even more preferably, for example, a pharmaceutical composition comprises a compound or salt of the present invention with hydroxyethylcellulose 1%/Tween® 80 0.25%/antifoam 0.05% in deionized water. Most preferably, the hydroxyethylcellulose is Natrosol® 250 L Pharm and the antifoam is DOW CORNING® ANTIFOAM 1510—US. Optionally, the composition further comprises nicotinic acid.

The compounds of the present invention are generally effective over a wide dosage range. For example, dosages per day normally fall within the daily range of about 1-1000 mg. Preferably such doses fall within the daily range of 25-400 mg. More preferably such doses fall within the daily range of 100-120 mg. Additionally, dosages per day of nictotinic acid, for example, NIASPAN® (slow release nicotinic acid), if necessary, normally fall within the range of about 50-2000 mg/day. In some instances dosage levels below the lower limit of the aforesaid ranges may be more than adequate, while in other cases still larger doses may be employed, and therefore the above dosage ranges are not intended to limit the scope of the invention in any way. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

We claim:

1. A compound of the following formula: 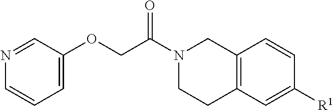
wherein:
R¹ is —NHSO₂R², —NHC(O)CH₂R³, —CH₂-piperazinyl-C(O)R⁴, or —CH(CH₃)-piperazinyl-C(O)R⁴;
R² is N-methylpiperidin-4-yl, N-oxetan-3-yl-piperidin-4-yl, tetrahydropyran-4-yl, tetrahydropyran-4-yl-N-carbonyl-piperidin-4-yl, 2-hydroxy-2-methyl-prop-1-yl, methoxyethyl, 2-isopropoxyethyl, 2-trifluoromethylethyl, cyclopropylmethyl, or pyrid-2-yl;
R³ is tetrahydropyran-2-yl, t-butyl, —CH(CH₃)(CH₃)(OH), —C(OH)(CH₃)(CH₂CH₃), or —C(OH)(CH₃)(CF₃); and
R⁴ is tetrahydropyran-4-yl, tetrahydropyran-4-yl-methyl, morpholin-4-yl-methyl, or 2-hydroxy-2-methyl-propyl;
or a pharmaceutically acceptable salt thereof.

2. The compound according to claim 1 wherein R¹ is —NHSO₂R².

3. The compound according to claim 1 which is 2-hydroxy-2-methyl-N-[2-[2-(3-pyridyloxy)acetyl]-3,4-dihydro-1H-isoquinolin-6-yl]propane-1-sulfonamide, or a pharmaceutically acceptable salt thereof.

4. The compound according to claim 1 which is 2-methoxy-N-[2-[2-(3-pyridyloxy)acetyl]-3,4-dihydro-1H-isoquinolin-6-yl]ethanesulfonamide, or a pharmaceutically acceptable salt thereof.

5. A pharmaceutical composition comprising a of the following formula: 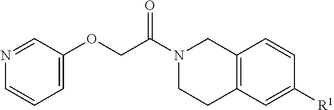
wherein:
R¹ is —NHSO₂R², —NHC(O)CH₂R³, —CH₂-piperazinyl-C(O)R⁴, or —CH(CH₃)-piperazinyl-C(O)R⁴;
R² is N-methylpiperidin-4-yl, N-oxetan-3-yl-piperidin-4-yl, tetrahydropyran-4-yl, tetrahydropyran-4-yl-N-carbonyl-piperidin-4-yl, 2-hydroxy-2-methyl-prop-1-yl, methoxyethyl, 2-isopropoxyethyl, 2-trifluoromethylethyl, cyclopropylmethyl, or pyrid-2-yl;
R³ is tetrahydropyran-2-yl, t-butyl, —CH(CH₃)(CH₃)(OH), —C(OH)(CH₃)(CH₂CH₃), or —C(OH)(CH₃)(CF₃); and
R⁴ is tetrahydropyran-4-yl, tetrahydropyran-4-yl-methyl, morpholin-4-yl-methyl, or 2-hydroxy-2-methyl-propyl; or a pharmaceutically acceptable salt thereof;
and one or more pharmaceutically acceptable carriers, diluents, or excipients.

6. The pharmaceutical composition according to claim 5 further comprising nicotinic acid.

7. A method of treating cancer in a mammal comprising administering to a mammal in need of such treatment an effective amount of a compound of the following formula: 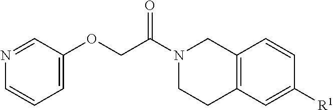
wherein:
R¹ is —NHSO₂R², —NHC(O)CH₂R³, —CH₂-piperazinyl-C(O)R⁴, or —CH(CH₃)-piperazinyl-C(O)R⁴;
R² is N-methylpiperidin-4-yl, N-oxetan-3-yl-piperidin-4-yl, tetrahydropyran-4-yl, tetrahydropyran-4-yl-N-carbonyl-piperidin-4-yl, 2-hydroxy-2-methyl-prop-1-yl, methoxyethyl, 2-isopropoxyethyl, 2-trifluoromethylethyl, cyclopropylmethyl, or pyrid-2-yl;
R³ is tetrahydropyran-2-yl, t-butyl, —CH(CH₃)(CH₃)(OH), —C(OH)(CH₃)(CH₂CH₃), or —C(OH)(CH₃)(CF₃);
R⁴ is tetrahydropyran-4-yl, tetrahydropyran-4-yl-methyl, morpholin-4-yl-methyl, or 2-hydroxy-2-methyl-propyl; or a pharmaceutically acceptable salt thereof;
wherein the cancer is selected from the group comprising breast cancer, gastric cancer, colorectal cancer, liver cancer, renal cancer, brain cancer, melanoma, prostate cancer, ovarian cancer, NSCLC, sarcoma, glioblastoma, neuroblastoma, leukemia, lymphoma, endometrial, kidney, adrenal gland, and autonomic ganglia cancers.

8. The method according to claim 7 wherein the cancer is ovarian cancer.

9. The method according to claim 7 wherein the cancer is NSCLC.

10. The method according to claim 7 wherein the cancer is lymphoma.

11. The method according to claim 7 wherein the compound or the salt thereof is administered in simultaneous, separate, or sequential combination with nicotinic acid.

12-18. (canceled)
